(19) United States
(12) Patent Application Publication
(10) Pub. No.: US 2015/0209342 A1
(43) Pub. Date: Jul. 30, 2015

(54) TOPICAL RETINOID FORMULATIONS, PROCESSES FOR MAKING AND METHODS OF USE

(71) Applicant: Allergan, Inc., Irvine, CA (US)

(72) Inventors: Guang Wei Lu, Irvine, CA (US); Fangjing Wang, Irvine, CA (US); Shaoxin Feng, Tustin, CA (US); Hui Liu, Irvine, CA (US); Patrick Hughes, Aliso Viejo, CA (US); Scott Smith, Mission Viejo, CA (US); Rong Yang, Mission Viejo, CA (US)

(21) Appl. No.: 14/607,473

(22) Filed: Jan. 28, 2015

Related U.S. Application Data
(60) Provisional application No. 61/932,564, filed on Jan. 28, 2014.

(51) Int. Cl. A61K 31/4436; A61K 31/192; A61K 31/203

(52) U.S. Cl. CPC A61K 31/4436; A61K 31/203; A61K 31/192

(57) ABSTRACT

The present invention provides topical dermal compositions including the compositions of the invention are useful for treating a variety of conditions associated with excess sebum production, such as, for example, acne.

### CROSS-REFERENCE TO RELATED APPLICATIONS

This application is a non-provisional application which claims the benefit of U.S. provisional application 61/932,564 entitled “Topical Retinoid Formulations, Processes For Making And Methods Of Use” filed on Jan. 28, 2014 with docket number 19348PROV (AP) which is incorporated herein by reference in its entirety and serves as the basis for a benefit and/or priority claim of the present application.

### FIELD

The present invention relates to topically administered dermal formulations such as formulations which comprise biodegradable microparticles containing a retinoid and which formulations are useful to treat a variety of skin conditions, disease or disorders.

### BACKGROUND

Human skin is composed of three primary layers: the stratum corneum, the epidermis, and the dermis. The outer layer is the stratum corneum. Its primary function is to serve as a barrier to the external environment. Lipids are secreted to the surface of the stratum corneum, where they decrease the stratum corneum's water permeability. Sebum typically constitutes 95% of these lipids. Abramovits et al., Dermatologic Clinics, 18:4 (2000). In addition to maintaining the epidermal permeability barrier, sebum transports anti-oxidants to the surface of the skin and protects against microbial colonization.

Sebum is produced in the sebaceous glands. These glands are present over most of the surface of the body. The highest concentration of these glands occurs on the scalp, the forehead, and the face. Despite the important physiological role that sebum plays, many individuals experience excess sebum production, especially in the facial area. An increased rate of sebum excretion is termed seborrhoea.

Seborrhoeic dermatitis is also associated with seborrhea. The condition is characterized by the appearance of red, flaking, greasy areas of skin, most commonly on the scalp, nasolabial folds, ears, eyebrows and chest. In the clinical literature seborrhoeic dermatitis may be also referred to as “sebopsoriasis,” “seborrhoeic eczema,” “dandruff,” and “pityriasis capitis.” Yeast infections are a causative factor in seborrhoeic dermatitis. The yeast thrives on sebum and leaves high concentrations of unsaturated fatty acids on the skin, thereby irritating it.

Acne vulgaris is associated with clinical seborrhea and there is a direct relationship between the sebum excretion rate and the severity of acne vulgaris. Although sebum production increases during adolescence (particularly in boys, because of androgen stimulation), increased sebum alone does not cause acne. Bacteria, most importantly P. acnes, feed on sebum and as a result are present in increased numbers in persons who have acne. Much of the inflammation associated with acne arises from the action of enzymes produced by the bacteria.

Acne vulgaris is characterized by areas of skin with seborrhea (scaly red skin), comedones (blackheads and whiteheads), papules (pinheads), pustules (pimples), nodules (large papules), and in more severe cases, scarring. It mostly affects skin with the densest population of sebaceous follicles, such as the face, upper chest, and back.

There are four key pathogenic factors of acne:
Follicular hyperkeratinization
Propionibacterium acnes (P. acnes)
Inflammation
Excessive sebum production (seborrhea)

Acne is still a very underserved market with treatment options that are only marginally effective. Only one product, oral ACCUTANE® (isotretinoin) that reduces sebum production has been highly effective, but at the expense of a black box warning with significant side effects including teratogenicity that require extensive patient monitoring. ACCUTANE® is indicated only for acne which is severe and recalcitrant to other treatment

Topical therapy is often preferred over oral therapy because of the reduced risk for adverse systemic effects. The most common topical drugs for acne can be divided into the following categories:
Retinoids (i.e., tazarotene, tretinoin, adapalene)
Antibiotics (i.e., clindamycin)
Benzoyl peroxide (BPO)
Others (i.e., dapsone, azelaic acid)

While many topical therapies are available, none of them address all four factors and most specialize in a few of these factors. Currently, no topical therapies in the market address excessive sebum production. Sebum is produced by the sebaceous gland, which is an appendage of the hair follicle, so it makes sense to target the sebaceous gland for more effective therapy. Since P. acnes depends on sebum to live, reduction of sebum is also thought to indirectly reduce P. acnes.

### Retinoids

Topical retinoids primarily act by normalizing infundibular hyperkeratinization and reducing inflammation, hence topical retinoids remain a mainstay for treatment of mild-to-moderate acne. The current topical retinoid formulations do not inhibit sebum production and their use is often limited by local tolerability (i.e., skin irritation).

Retinoids are compounds related to vitamin A. The formula for vitamin A is:

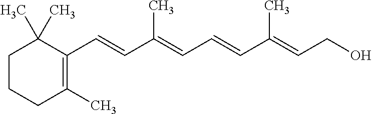 [SMILES: CC1=C(/C=C/C(C)=C/C=C/C(C)=C/CO)C(C)(C)CCC1]

Examples of known retinoids include retinol, tretinoin, isotretinoin, etretinate, acitretin and tazarotene. The formulas for retinol, tretinoin, isotretinoin, etretinate, and acitretin are each shown below.

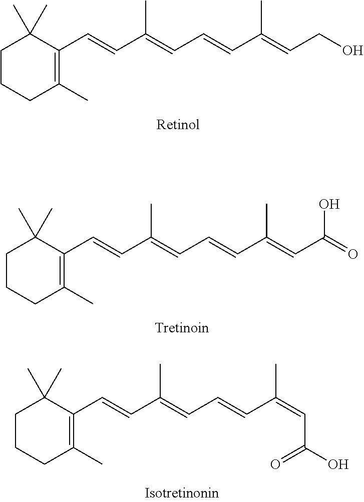 [SMILES: CC1=C(/C=C/C(C)=C/C=C/C(C)=C/CO)C(C)(C)CCC1 ; CC1=C(/C=C/C(C)=C/C=C/C(C)=C/C(=O)O)C(C)(C)CCC1 ; CC1=C(/C=C/C(C)=C/C=C/C(C)=C\C(=O)O)C(C)(C)CCC1]

### Second Generation

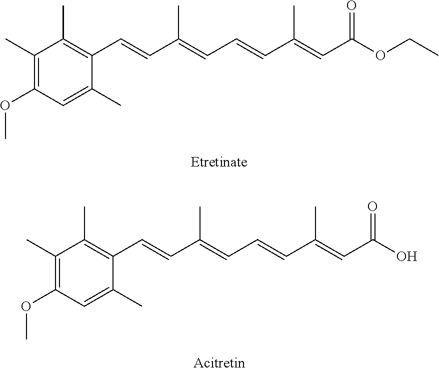

It is generally recognized that there are three generations of retinoids. First generation retinoids include retinol, retinal, tretinoin (retinoic acid, Retin-A), isotretinoin, and alitretinoin, second generation retinoids include etretinate and its metabolite acitretin and third generation retinoids include tazarotene, bexarotene and adapalene. Various retinoids have been used in as topical treatments of different conditions including acne, psoriasis, and photoaging.

### Tazarotene

Tazarotene has the structural formula:

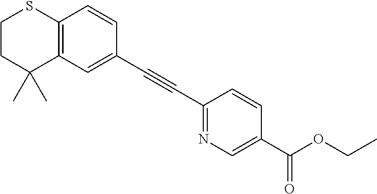 [SMILES: CCOC(=O)c1ccc(C#Cc2ccc3c(c2)C(C)(C)CCS3)nc1]

Tazarotene has the IUPAC name ethyl 6-[2-(4,4-dimethyl-3,4-dihydro-2H-1-benzothiopyran-6-yl)ethynyl)]pyridine-3-carboxylate and abbreviated formula C₂₁H₂₁NO₂S. The molecular weight of tazarotene is 351 (molecular mass is 351.463 g/mol).

Tazarotene is a retinoid prodrug converted to its active form a carboxylic acid of tazarotene by deesterification. Tazarotenic acid binds to all the retinoic acid cellular receptors RARα, RARβ, and RARγ. Pharmokinetic studies have shown that tazarotene has a half life (once topically applied, released from its cream, emulsion, gel, solution, emulsion, etc formulation, and after receptor binding or entry into the circulation).

Tazarotene has been sold as a topically applied cream under the various trade names Tazorac, Avage and Zorac. Tazarotene is a retinoid, specifically tazarotene an acetylenic retinoid and has been used for the treatment of psoriasis, acne, and sun damaged skin (photodamage), usually in 0.05% and 0.1% concentrations. Side effects of tazarotene topical application include a worsening of acne, increased sensitivity to sunlight, dry skin, itchiness, redness (erthema), and skin drying and cracking.

Typically microparticles have a diameter between about 0.1 micron and 100 microns in size. Commercially available microparticles are available in a wide variety of materials, including ceramics, glass, polymers, and metals. Microspheres are spherical microparticles.

Tazarotene microspheres for intraocular use are discussed in published US patent applications US 2011/0076318 A1 and US 2012/0157499 A1, the entire contents of which are incorporated herein by reference. K Mader. Resomer®-Biodegradable polymers for sutures, medical devices, drug delivery systems and tissue engineering. Aldrich.com 2012 discusses various resomers and biodegradable polymers. A Park et al. Microparticle and liquid formulation of novel HIV protease inhibitor. Pharm Dev Tech (2002) 7:297-303, discusses certain microparticles. P O'Donnell and J McGinity. Preparation of microspheres by the solvent evaporation technique. Advanced Drug Delivery Reviews (1997) 28: 25-42, discusses a method for preparing certain microspheres. U.S. patent application Ser. No. 13/486,137, published as US 2012/0328670 and entitled “Targeted delivery of retinoid compounds to the sebaceous glands” discusses certain tazarotene containing microspheres.

Thus there is a need for topical retinoid compositions that can provide extended or sustained release (as opposed to immediate topical release or delivery) of the retinoid in therapeutically effect amounts (such as treating acne by reducing sebum production) with reduced side effects as compared to known retinoid topical treatments.

### SUMMARY

The present invention provides topical dermal compositions including microsphere encapsulated retinoids. Pharmaceutically acceptable salts, esters, or amides of a retinoid are also contemplated for use in the practice of the invention. The compositions of the invention are useful for treating a variety of conditions associated with excess sebum production, such as, for example, acne.

By employing the compositions and methods of the invention, a retinoid can be delivered deep into hair follicles where it can reach sebaceous gland to treat acne.

The present invention includes a topical dermal composition comprising a plurality of biodegradable polymeric microparticles; a retinoid contained by the microparticles, and; a vehicle for the microparticles comprising an aqueous solvent and a non-aqueous solvent. The non-aqueous solvent can be selected from the group consisting of glycerin, propylene glycol, ethanol and transcutol. Additionally, the vehicle is preferably about 70% glycerin and about 30% saline. The retinoid can be selected from the group consisting of tretinoin, adapalene, and tazarotene, combination, salts and esters thereof and the microparticles can have an average diameter of between about 1 micron and about 10 microns and preferably have an average diameter of between about 2 microns and about 7 microns. The biodegradable polymer use to make the microparticles can be selected from the group consisting of polymeric lactic acid, polymeric glycolic acid, and polymeric lactic acid glycolic acid (“PLGA”), and combinations thereof, or the biodegradable polymer can be a PLA. The retinoid loading in the microspheres can be between about 1% and about 15% and is preferably about 15% to about 30%.

An embodiment of the present invention is topical dermal composition comprising a plurality of biodegradable PLGA or PLA microparticles having an average diameter of between about 2 microns and about 7 microns; tazarotene contained by the microparticles at about 30% drug loading, and; a vehicle for the microparticles comprising saline and glycerin.

The present invention also encompasses a method for treating a dermatological condition selected from the group consisting of acne vulgaris, seborrhoeic dermatitis, psoriasis, keratosis pilaris, and photoaged skin, the method comprising the step of administering to the skin of a person with a dermatological condition:

a topical dermal composition comprising a plurality of biodegradable polymeric microparticles;

a retinoid contained by the microparticles; and

a vehicle for the microparticles comprising an aqueous solvent and a non-aqueous solvent, to thereby permit treating the dermatological condition.

The composition can provide for an extended release of the retinoid.

The invention also includes a method for treating acne, the method comprising the step of administering to the skin of a person with acne:

a topical dermal composition comprising a plurality of biodegradable PLGA microparticles having an average diameter of between about 2 microns and about 7 microns, wherein tazarotene is contained by the microparticles at about 30% drug loading, and;

a vehicle for the microparticles comprising saline and glycerin, thereby permitting treatment the acne.

In another embodiment the present invention encompasses a dermal topical composition comprising:

a plurality of biodegradable polymeric microparticles;

a vehicle for the microparticles comprising an aqueous solvent and a non-aqueous solvent; and,

a compound of the following formula contained by or encapsulated by the microparticles:

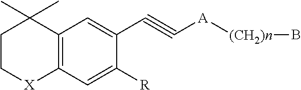

wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
   and wherein the microparticles have an average diameter between about 0.1 μm and about 10 μm.

The microparticles can have an average diameter no greater than about 5 μm, an average diameter no greater than about 4 μm or an average diameter no greater than about 1 μm. The biodegradable polymer can be selected from the group consisting of poly hydroxyaliphatic carboxylic acids, polyesters, polysaccharides, and combinations thereof. The biodegradable polymer can be poly(lactic-co-glycolic acid) (PLGA) and the compound can be tazarotene or tazarotenic acid or a pharmaceutically acceptable salt, ester or amide thereof.

The invention also encompasses a method for treating a condition associated with excess sebum production, the method comprising the step of topically applying to the skin of a patient in need of such treatment a dermal composition comprising:

(1) a plurality of biodegradable polymeric micronanoparticles;

(2) encapsulated by or encompassed by the microparticles a compound of the formula:

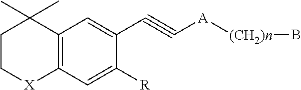

or a pharmaceutically acceptable salt thereof, wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
   and;

(3) a vehicle for the microparticles comprising an aqueous solvent and a non-aqueous solvent;

wherein the microparticles have an average diameter between about 0.1 μm and about 10 μm; and wherein the compound penetrates the hair follicle to the depth of the sebaceous gland, and acts directly on the gland to reduce sebum production by the gland.

The invention also encompasses a method for treating a condition associated with excess sebum production, the method comprising the step of topically applying to the skin of a patient in need of such treatment a dermal composition comprising:

(1) plurality of biodegradable, polymeric microparticles

(2) encapsulated by or encompassed by the microparticles a compound of the formula:

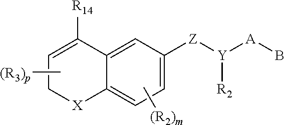

or a pharmaceutically acceptable salt thereof, wherein:
X is S, O, NR′ wherein R′ is H or alkyl of 1 to 6 carbons, or X is [C(R₁)₂]ₙ where R₁ is independently H or alkyl of 1 to 6 carbons, and n is an integer between, and including, 0 and 2;
R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons;
R₃ is hydrogen, lower alkyl of 1 to 6 carbons or F;
m is an integer having the value of 0-3;
p is an integer having the value of 0-3;
Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—; —CSO—; —OCS—;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O, or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons; and;

(3) a vehicle for the microparticles comprising an aqueous solvent and a non-aqueous solvent;

wherein the microparticles have an average diameter between about 0.1 μm and about 10 μm; and wherein the compound penetrates the hair follicle to the depth of the sebaceous gland, and acts directly on the gland to reduce sebum production by the gland.

### DRAWINGS

FIGS. 1A-1H show a collection of eight SEM images taken of PLGA Tazarotene microspheres dispersed in phosphate buffered saline (“PBS”) and/or glycerin vehicles for 1 month. The magnification was 2000× using a ZEISS EVO® 40 SEM (scanning electron microscope) set at an acceleration voltage of 5.0 kV. The labels at the bottom of each of the eight FIG. 1 SEM image indicates the specific polymer used to make MS, and the vehicles used for the degradation study. (see Example 1). In FIG. 1A, Pa1=7.788 μm and Pa2=4.272 μm. In FIG. 1B, Pa1=10.99 μm and Pa2=10.12 μm. In FIG. 1C, Pa1=9.103 μm and Pa2=7.688 μm. In FIG. 1D, Pa1=5.421 μm and Pa2=8.920 μm. In FIG. 1E, Pa1=9.711 μm and Pa2=22.82 μm. In FIG. 1F, Pa1=9.991 μm and Pa2=9.524 μm. In FIG. 1G, Pa1=16.78 μm and Pa2=14.37 μm. In FIG. 1H, Pa1=52.53 and Pa2=9.319 μm.

FIG. 2 shows a graph showing on the x axis the time in days (up to 28 days), and on the y axis the peak molecular weight of RG503H polymer tazarotene microspheres stored for one month in either 70% glycerin vehicle or in PBS vehicle, at either 25 or at 40 degrees C.

FIG. 3 shows a graph showing on the x axis the time in days (up to 28 days) and on the y axis the peak molecular weight of RG203H/RG755S polymer mix tazarotene microspheres stored for one month in either 70% glycerin vehicle or in PBS vehicle, at 40 degrees C.

FIG. 4 shows a graph showing on the x axis the time in days (up to 28 days) and on the y axis the peak molecular weight of R208 polymer tazarotene microspheres stored for one month in either 70% glycerin vehicle or in PBS vehicle, at 40 degrees C.

FIG. 5 shows a graph showing on the x axis the time in days (up to 48 hours) and on the y axis % tazarotene released from R203H/RG755S polymer tazarotene loaded microspheres in either PHS or 70% glycerin vehicle maintained at either 5 or 25 degrees C.

FIG. 6 shows a graph showing on the x axis time in hours (up to 168 hours) and on the y axis cumulative release over time of the tazarotene (at four different tazarotene loading amounts) from the R208 polymer microspheres.

### DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

The invention provides topical dermal compositions including a plurality of microparticles, wherein the particles include a biodegradable polymer and a retinoid wherein the particles have an average diameter between about 0.1 μm and about 10 μm. In some embodiments, the particles have an average diameter no greater than about 5 μm. In some embodiments, the particles have an average diameter no greater than about 4 μm. In some embodiments, the particles have an average diameter no greater than about 1 μm. Biodegradable polymers contemplated for use in the practice of the invention include, but are not limited to, poly hydroxyaliphatic carboxylic acids, polyesters, polysaccharides, and combinations thereof. In some embodiments, the biodegradable polymer is poly(lactic-co-glycolic acid) (PLGA).

The term “ester” refers to any compound falling within the definition of that term as classically used in organic chemistry. It includes organic and inorganic esters. Unless stated otherwise in this application, esters are derived from the saturated aliphatic alcohols or acids of ten or fewer carbon atoms or the cyclic or saturated aliphatic cyclic alcohols and acids of 5 to 10 carbon atoms. Examples include aliphatic esters derived from lower alkyl acids and alcohols, and phenyl or lower alkyl phenyl esters.

The term “amide” has the meaning classically accorded that term in organic chemistry. In this instance it includes the unsubstituted amides and all aliphatic and aromatic mono- and di-substituted amides. Examples include the mono- and di-substituted amides derived from the saturated aliphatic radicals of ten or fewer carbon atoms or the cyclic or saturated aliphatic-cyclic radicals of 5 to 10 carbon atoms. In one embodiment, the amides are derived from substituted and unsubstituted lower alkyl amines. In another embodiment, the amides are mono- and disubstituted amides derived from the substituted and unsubstituted phenyl or lower alkylphenyl amines. One may also use unsubstituted amides.

“Acetals” and “ketals” include the radicals of the formula-CK where K is (—OR)₂. Here, R is lower alkyl. Also, K may be —OR₇O— where R₇ is lower alkyl of 2-5 carbon atoms, straight chain or branched.

The term “alkyl,” by itself or as part of another substituent, means, unless otherwise stated, a straight (i.e. unbranched) or branched carbon chain, or combination thereof, which may be fully saturated (referred to herein as a “saturated alkyl”), mono- or polyunsaturated and can include di- and multivalent radicals, having the number of carbon atoms designated (e.g. “C₁-C₁₀” means one to ten carbons). Typical alkyl groups include, for example, methyl, ethyl, propyl, isopropyl, butyl, isobutyl, tertiary butyl, pentyl, hexyl and the like. The term “lower alkyl” refers to a C1-C6 alkyl group (e.g. methy, ethyl, propyl, isopropyl, n-butyl, sec-butyl, tert-butyl, pentyl, hexyl, and others identifiable to a skilled person). An “alkoxy” is an alkyl attached to the remainder of the molecule via an oxygen linker (—O—).

The term “aryl” means, unless otherwise stated, an aromatic substituent of 3 to 14 atoms (e.g. 6 to 10) which can be a single ring or multiple rings (e.g., from 1 to 3 rings) which may be fused together (i.e. a fused ring aryl) or linked covalently. A fused ring aryl refers to multiple rings fused together wherein at least one of the fused rings is an aryl ring (e.g., phenyl, 1-naphthyl, 2-naphthyl, or 4-biphenyl). The term “heteroaryl” refers to aryl groups (or rings) that contain one or more (e.g., 4) heteroatoms selected from N, O, and S, wherein the nitrogen and sulfur atoms are optionally oxidized, and the nitrogen atom(s) are optionally quaternized, the remaining ring atoms being carbon. The heteroaryl may be a monovalent monocyclic, bicyclic, or tricyclic (e.g., monocyclic or bicyclic) aromatic radical of 5 to 14 (e.g., 5 to 10) ring atoms where one or more, (e.g., one, two, or three or four) ring atoms are heteroatom selected from N, O, or S.

The terms “cycloalkyl” and “heterocycloalkyl,” by themselves or in combination with other terms, represent, unless otherwise stated, non-aromatic cyclic versions of “alkyl” and “heteroalkyl”, respectively (e.g., having 4 to 8 ring atoms). Additionally, for heterocycloalkyl, a heteroatom can occupy the position at which the heterocycle is attached to the remainder of the molecule.

Pharmaceutically acceptable salts of retinoids are also contemplated for use in the practice of the invention. A pharmaceutically acceptable salt is any salt which retains the activity of the parent compound and does not impart any deleterious or untoward effect on the subject to which it is administered and in the context in which it is administered.

Pharmaceutically acceptable acid addition salts of a retinoid are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts.

Pharmaceutically acceptable salts may be derived from organic or inorganic bases. The salt may be a mono or polyvalent ion. Of particular interest are the inorganic ions, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Where there is a nitrogen sufficiently basic as to be capable of forming acid addition salts, such may be formed with any inorganic or organic acids or alkylating agent such as methyl iodide. Preferred salts are those formed with inorganic acids such as hydrochloric acid, sulfuric acid or phosphoric acid. Any of a number of simple organic acids such as mono-, di- or tri- acid may also be used.

The particles included in the compositions of the invention have an average diameter no less than about 0.1 μm and no greater than about 10 μm

In one embodiment, the particle is shaped like a sphere. The inventors refer to such particles as “microspheres,” even though they may have an average diameter in the nanometer range (that is, about 100 nm to about 999 nm). The microspheres of the invention have a maximum average diameter of about 10 μm.

As used here, the term “about,” when used in connection with a value, means that the value may not differ by more than 5%. Hence, “about 10 μm” includes all values within the range of 9.5 μm to 10.5 μm.

In one embodiment, the microspheres of the invention have a maximum average diameter of about 10 μm. In another embodiment, the microspheres of the invention have a maximum average diameter of about 9 μm. In another embodiment, the microspheres of the invention have a maximum average diameter of about 8 μm. In another embodiment, the microspheres of the invention have a maximum average diameter of about 7 μm. In another embodiment, the microspheres of the invention have a maximum average diameter of about 6 μm. In another embodiment, the microspheres of the invention have a maximum average diameter of about 5 μm. In another embodiment, the microspheres of the invention have a maximum average diameter of about 4 μm. In another embodiment, the microspheres of the invention have a maximum average diameter of about 3 μm. In another embodiment, the microspheres of the invention have a maximum average diameter of about 2 μm. In another embodiment, the microspheres of the invention have a maximum average diameter of about 1 μm.

In another embodiment, the microspheres of the invention have a maximum average diameter less than about 1 μm. In another embodiment, the microspheres of the invention have a maximum average diameter of about 0.9 μm. In another embodiment, the microspheres of the invention have a maximum average diameter of about 0.8 μm. In another embodiment, the microspheres of the invention have a maximum average diameter of about 0.7 μm. In another embodiment, the microspheres of the invention have a maximum average diameter of about 0.6 μm. In another embodiment, the microspheres of the invention have a maximum average diameter of about 0.5 μm. In another embodiment, the microspheres of the invention have a maximum average diameter of about 0.4 μm. In another embodiment, the microspheres of the invention have a maximum average diameter of about 0.3 μm. In another embodiment, the microspheres of the invention have a maximum average diameter of about 0.2 μm. In another embodiment, the microspheres of the invention have a maximum average diameter of about 0.1 μm.

In one embodiment, the particle is shaped like a cylindrical rod. The disclosure refers to such particles as “microcylinders,” even though they may have an average diameter in the nanometer range (that is, about 100 nm to about 999 nm). The microcylinders of the invention have a maximum average diameter and maximum average length such that no one such dimension is greater than about 10 μm. In other embodiments, the particles of the invention are of different geometry, such as fibers or circular discs; any geometry falls within the scope of the invention, as long as the average of any single dimension of the particle exceeds about 10 μm.

In one embodiment, the microcylinders of the invention have a maximum average diameter of about 10 μm. In another embodiment, the microcylinders of the invention have a maximum average diameter of about 9 μm. In another embodiment, the microcylinders of the invention have a maximum average diameter of about 8 μm. In another embodiment, the microcylinders of the invention have a maximum average diameter of about 7 μm. In another embodiment, the microcylinders of the invention have a maximum average diameter of about 6 μm. In another embodiment, the microcylinders of the invention have a maximum average diameter of about 5 μm. In another embodiment, the microcylinders of the invention have a maximum average diameter of about 4 μm. In another embodiment, the microcylinders of the invention have a maximum average diameter of about 3 μm. In another embodiment, the microcylinders of the invention have a maximum average diameter of about 2 μm. In another embodiment, the microcylinders of the invention have a maximum average diameter of about 1 μm.

In another embodiment, the microcylinders of the invention have a maximum average diameter less than about 1 μm. In another embodiment, the microcylinders of the invention have a maximum average diameter of about 0.9 μm. In another embodiment, the microcylinders of the invention have a maximum average diameter of about 0.8 μm. In another embodiment, the microcylinders of the invention have a maximum average diameter of about 0.7 μm. In another embodiment, the microcylinders of the invention have a maximum average diameter of about 0.6 μm. In another embodiment, the microcylinders of the invention have a maximum average diameter of about 0.5 μm. In another embodiment, the microcylinders of the invention have a maximum average diameter of about 0.4 μm. In another embodiment, the microcylinders of the invention have a maximum average diameter of about 0.3 μm. In another embodiment, the microcylinders of the invention have a maximum average diameter of about 0.2 μm. In another embodiment, the microcylinders of the invention have a maximum average diameter of about 0.1 μm.

In one embodiment, the microcylinders have a maximum average length of about 10 μm, about 9 μm, about 8 μm, about 7 μm, about 6 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, or about 0.2 μm.

The size and geometry of the particle can also be used to control the rate of release, period of treatment, and drug concentration. Larger particles will deliver a proportionately larger dose, but, depending on the surface to mass ratio, may have a slower release rate.

The retinoid of the invention can be in a particulate or powder form. In one embodiment, the retinoid itself consists of particles having the dimensions described above.

In another embodiment, the retinoid is combined with a biodegradable polymer. In one embodiment, retinoid is from about 10% to about 90% by weight of the composition. In another embodiment, retinoid is from about 20% to about 80% by weight of the composition. In another embodiment, the retinoid is from about 30% to about 70% by weight of the composition. In another embodiment, the retinoid is from about 40% to about 60% by weight of the composition. In one embodiment, the retinoid comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the composition.

Suitable polymeric materials for use in the compositions of the invention include those materials which are biocompatible with the skin so as to cause no substantial irritation or other side effects. In one embodiment, such materials are at least partially biodegradable. In another embodiment, such materials are completely biodegradable.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials can be addition or condensation polymers, advantageously condensation polymers. The polymeric materials can be cross-linked or non-cross-linked, for example not more than lightly cross-linked, such as less than about 5%, or less than about 1% of the polymeric material being cross-linked. In some embodiments, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen can be present as amide, cyano and amino. The polymers set forth in Heller, CRC Critical Reviews in Therapeutic Drug Carrier Systems, Vol. 1, CRC Press, Boca Raton, Fla. 1987, pp 39-90 (Biodegradable Polymers in Controlled Drug Delivery), the contents of which are incorporated herein by reference, which describes encapsulation for controlled drug delivery, may find use in the present compositions.

Additional polymers include, for example, polymers of hydroxyaliphatic carboxylic acids, either homopolymers or copolymers, and polysaccharides, lipid nanoparticle, and mesoporous silica nanoparticle. Exemplary polyesters include, for example, polymers of D-lactic acid, L-lactic acid, racemic lactic acid, glycolic acid, polycaprolactone, and combinations thereof. Generally, by employing the L-lactate or D-lactate, a slowly eroding polymer or polymeric material can be achieved, while erosion is substantially enhanced with the lactate racemate.

Exemplary polysaccharides include, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some desirable characteristics of the polymers or polymeric materials for use in the present invention can include, for example, biocompatibility, compatibility with the therapeutic compound, ease of use of the polymer in making the compositions of the present invention, a half-life in the physiological environment of at least about 6 hours, preferably greater than about one day, and water insolubility.

The biodegradable polymeric materials which are included to form the particles are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the system is the relative average molecular weight of the polymeric composition employed in the system. Different molecular weights of the same or different polymeric compositions may be included in the system to modulate the release profile. In certain systems, the relative average molecular weight of the polymer will range from about 9 to about 64 kD, from about 10 to about 54 kD, or from about 12 to about 45 kD.

In some compositions, copolymers of glycolic acid and lactic acid (poly(lactic-co-glycolic acid)) are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the drug delivery system, where a more flexible system is desirable for larger geometries. The proportion of polylactic acid in the polylactic acid-polyglycolic acid (PLGA) copolymer can be 0-100%; in other embodiments, the proportion of polylactic acid can be from about 10% to about 90%, from about 20% to about 80%, from about 30% to about 70%,or from about 40% to about 60%. In one embodiment, the proportion of polylactic acid may be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the composition.

The biodegradable polymer of the composition of the invention can comprise a mixture of two or more biodegradable polymers. For example, the composition can comprise a mixture of a first biodegradable polymer and a different second biodegradable polymer. One or more of the biodegradable polymers can have terminal acid groups.

Release of a drug from an erodible polymer is the consequence of several mechanisms or combinations of mechanisms. Some of these mechanisms include, for example, desorption from the systems surface, dissolution, diffusion through porous channels of the hydrated polymer and erosion. Erosion can be bulk or surface or a combination of both.

One example of a composition of the invention includes a retinoid with a biodegradable polymer matrix that comprises a (lactide-co-glycolide) or a poly (D,L-lactide-co-glycolide). The composition system may have an amount of a retinoid from about 40% to about 70% by weight of the system.

The release of the retinoid from the composition can include an initial burst of release followed by a gradual increase in the amount of retinoid released, or the release can include an initial delay in release of retinoid followed by an increase in release. When the biodegradable polymer is substantially completely degraded, the percent of retinoid that has been released is about one hundred percent.

It can be desirable to provide a relatively constant rate of release of retinoid from the particles. However, the release rate can change to either increase or decrease depending on the formulation of the particle. In addition, the release profile of retinoid can include one or more linear portions and/or one or more non-linear portions. In one embodiment, the release rate is greater than zero once the system has begun to degrade or erode.

The particles of the invention can be monolithic, that is, having the active agent or agents homogenously distributed through the polymer, or the can be encapsulated, where a reservoir of active agent is encapsulated by the polymer. Due to ease of manufacture, monolithic systems are usually preferred over encapsulated forms. However, the greater control afforded by the encapsulated, reservoir-type implants may be of benefit in some circumstances, where the therapeutic level of the drug falls within a narrow window. In addition, the retinoid may be distributed in a non-homogenous pattern in the polymer. For example, a particle may include a portion that has a greater concentration of the retinoid relative to a second portion of the implant.

Thus, particles can be prepared where the center may be of one material and the surface may have one or more layers of the same or a different material, where the layers may be cross-linked, or of a different molecular weight, different density or porosity, or the like. For example, where it is desirable to quickly release an initial bolus of drug, the center can be a polylactate coated with a polylactate-polyglycolate copolymer, so as to enhance the rate of initial degradation. Alternatively, the center can be polyvinyl alcohol coated with polylactate, so that upon degradation of the polylactate exterior the center would dissolve.

The proportions of retinoid, polymer, and any other modifiers can be empirically determined by formulating several drug delivery systems with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to a retinoid and polymer, the particles disclosed herein may include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents can be present in amounts sufficient to maintain a pH of the system of between about 2 to about 9, for example at about pH 4 to about 8. As such the buffering agent can be as much as about 5% by weight of the total drug delivery system. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from about 0.001% to about 5% by weight; in another embodiment, they may be present in amounts from about 0.01% to about 2% by weight.

In addition, the particles can include a solubility-enhancing compound provided in an amount effective to enhance the solubility of the retinoid relative to substantially identical systems without the solubility enhancing compound. For example, an implant can include a β-cyclodextrin, which is effective in enhancing the solubility of isotretinoin. The β-cyclodextrin can be provided in an amount from about 0.5% (w/w) to about 25% (w/w) of the particle. In other embodiments, the β-cyclodextrin is provided in an amount from about 5% (w/w) to about 15% (w/w) of the particle.

Additionally, release modulators such as those described in U.S. Pat. No. 5,869,079, the contents of which are incorporated herein by reference, can be included in the particles. The amount of release modulator employed will be dependent on the desired release profile, the activity of the modulator, and on the release profile of isotretinoin in the absence of modulator. Electrolytes such as sodium chloride and potassium chloride may also be included in the implant. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolve more slowly, slowing the exposure of drug particles, and thereby slowing the rate of drug bioerosion.

Various techniques can be employed to produce the microparticles described herein. In one embodiment, particles are produced using a solvent evaporation process. Such a process can include steps of liquid sieving, freeze drying, and sterilizing the various composition compounds. In one embodiment, isotretinoin and a polymer are combined with methylene chloride to form a first composition, and water and polyvinyl alcohol are combined to form a second composition. The first and second compositions are combined to form an emulsion. The emulsion is rinsed and/or centrifuged, and the resulting product dried. In a further embodiment, the emulsion undergoes an evaporation process to remove methylene chloride from the emulsion. For example, the emulsion can be evaporated for about 2 days or more. In this embodiment, the method includes sieving isotretinoin-containing microspheres in a liquid phase, as compared to a method which includes sieving retinoid-containing microparticles in a dry phase. This method can also comprise a step of freeze drying the sieved microparticles, and a step of packaging the freeze dried microparticles.

In another embodiment, a method of producing retinoid-containing microspheres includes one or more of the following steps, and in certain embodiments, the method includes each of the following steps: a polymeric material, such as PLGA, is dissolved in a solvent, such as methylene chloride. The dissolving of the PLGA can occur with stirring the mixture until the PLGA is completely dissolved. A predetermined amount of isotretinoin is added to the dissolved PLGA composition. The resulting composition can be understood to be a first composition in reference to this method. A second different composition is produced by combining heated water, for example water having a temperature of about 80° C., with polyvinylic alcohol (PVA). The PVA can be combined with the heated water by stirring the water at a rate effective in maintaining PVA in suspension without substantial bubble formation. The second composition may then be cooled to a desired temperature, such as room temperature.

An emulsion can be produced by combining the first composition and the second composition described in the preceding paragraph. For example, the second composition (i.e., the PVA solution) can be vigorously stirred while avoiding bubble formation. While stirring the second composition, the first composition is added to form an emulsion. As the mixture emulsifies, the stirring speed may be increased to keep the surface of the emulsion moving. Foam or bubble formation is minimized during these steps. In this method, the emulsion is stirred for at least two days (e.g., for about 48 hours or more). As the emulsion is stirred for about 24 hours, the emulsion begins to liquefy. To reduce the possibility of foaming, the stirring speed can be decreased as the emulsion liquefies. After about 48 hours, methylene chloride is substantially or completely evaporated. The method can include a step of determining the amount of methylene chloride in the evaporated material.

After the evaporation of the methylene chloride, the microparticle-containing composition is rinsed and sieved. For example, the microparticle-containing composition is combined with a liquid and centrifuged. The supernatant is removed and the pellet can be resuspended by sonication or other suitable method for additional centrifugation steps. After the microsphere suspension has been centrifuged, water can be added to rinse the microspheres, and the resulting supernatant can be removed by vacuum extraction. In some methods, at least three water rinsing steps are desirable. The rinsed pellets are then sieved through a plurality of filters. For example, the pellets can be passed through two superimposed filters having a pore size of about 125 μm and about 45 μm, respectively. The filters can be rinsed with water and the solution can be retrieved in the filter bottom.

The retrieved solution can then be combined with an additional amount of water and rinsed two or more times using a centrifuge. The rinsed pellet can then be placed in the filter bottom and covered with a filter to reduce loss of the microsphere material during a lyophilization process. The material is then frozen. For example, the material is frozen at −50° C. and freeze dried for at least twelve hours. After freeze drying, the microspheres can be stored in a package, and/or may be sterilized by a sterilization device, such as a source of gamma radiation.

Additional examples of methods for producing retinoid containing particles are described in U.S. Patent Application Publication No. 2011/0076318. Additional examples of producing particles of biodegradable polymer can be found in U.S. Patent Application Publication No. 2005/0003007 and No. 2008/0182909, the contents of both of which are incorporated herein by reference.

In one embodiment, the compositions of the invention can be used to treat conditions associated with excess sebum production. Such conditions include, for example, acne vulgaris, seborrhoeic dermatitis, and keratosis pilaris.

In another embodiment, the compositions of the invention can be used to treat those conditions in which it would be beneficial to suppress the function of the sebaceous gland. Such conditions include, for example, sebaceous cyst, sebaceous hyperplasia, sebaceous adenoma, and sebaceous gland carcinoma.

### EXAMPLES

The following Examples set forth details regarding certain embodiments of the present invention and are not intended to limit the invention.

In these Examples the microspheres (“MS”) described were made by emulsifying the identified polymers in a bead-loaded column followed by a solvent extraction process to remove the organic solvent. Additionally, in these Examples the MS studied comprised about 85% biodegradable polylactic polyglycolic polymer (“PLGA”) and/or polylactic acid (“PLA”) with for example about a 15% or 30% tazarotene drug loading. The RG503H (and RG503) PLGA polymers had a molecular weight range of 24,000-38,000, and an inherent viscosity (“IV”) of 0.32-0.44 (dl/g). The R203H (and R203) PLGA polymers had a molecular weight range of 18,000-28,000, and IV of 0.25-0.35 (dl/g). The RG575S (and RG575) PLGA polymers had a molecular weight of about 63,000, and an IV of 0.3-0.5 (dl/g). The R208H (and R208) PLA polymers had a molecular weight range of 180,000-200,000, and an IV of 1.8-2.2 (dl/g). The R502 PLGA polymers had a molecular weight range of 7,000-17,000, and an IV of 0.16-0.24 (dl/g).

### Example 1

### Suitable Vehicles for Sustained Delivery PLGA-Retinoid Microspheres

Sustained release retinoid formulations were developed. The formulations comprise the retinoid tazarotene contained within (for example by being encapsulated by and/or distributed within a polymeric matrix)) polymer (such as a PLGA) microparticles in a suitable vehicle (“vehicle” is synonymous with “carrier”). This Example 1 exemplifies studies carried out to determine suitable vehicles for the tazarotene microparticles. Exemplary suitable vehicles were determined to comprise: non-aqueous solvents (comprising from about 5% by weight to about 99% by weight of the formulation); surface active agents (comprising from about 0.05% to about 20% by weight of the formulation (suitable surface active agents include polysorbate 80 [Tween 80], poloxamer 407, poloxamer 188, sorbitan monooleate [Span 80], polysorbate 20, tyloxapol, polyglyceryl-3 methylglucose distearate, sodium cholate, polyoxyl 15 hydroxy stearate [sulotol HS 15], phosphatidylcholine, and soybean lecithin), and; viscosity agents (comprising from about 0.1% to about 20% by weight of the formulation (suitable viscosity agents include carbomers including carbopol 947, carbopol 2020, hydropropylmethyl cellulose [HPMC], hydropropyl cellulose [HPC], xanthum, chitosan, hydroxyethyl cellulose, and hydrixyethylmethylcellulose). In particular, such suitable vehicles can both stabilize the formulation and permit a desired controlled (sustained or extended) release of the tazarotene from the microparticles and out of the formulation to the site of desired therapeutic or cosmetic activity.

Thus tazarotene loaded polymeric polylactic polyglycolic acid (“PLGA”) microspheres (“MS”) were incorporated into a topical vehicle for deposition onto (application to) the skin. The vehicle can be, for example, a gel or a cream into which the tazarotene loaded microspheres are dispersed.

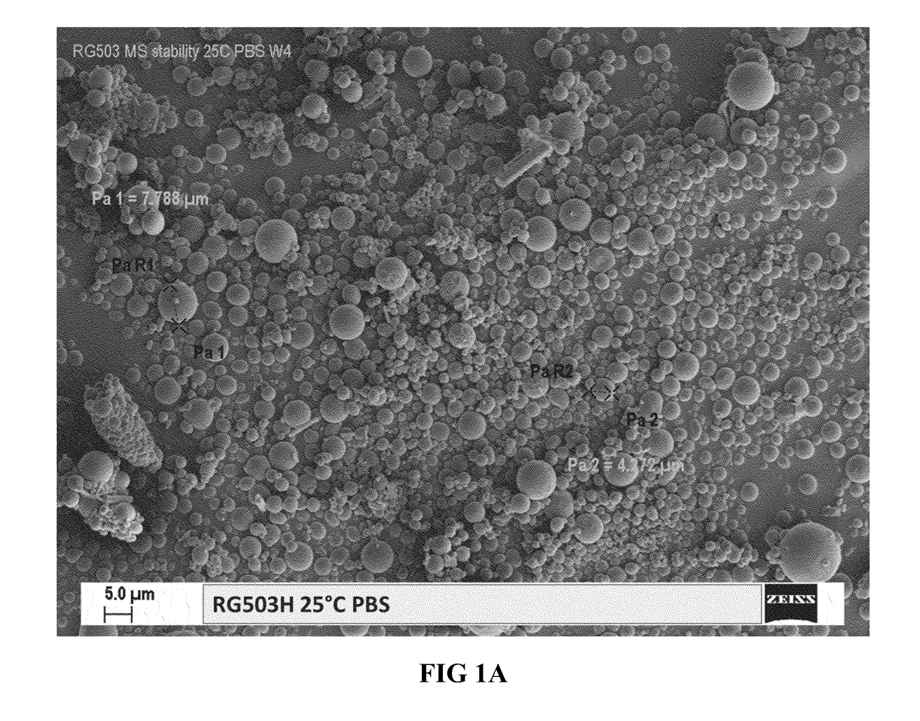

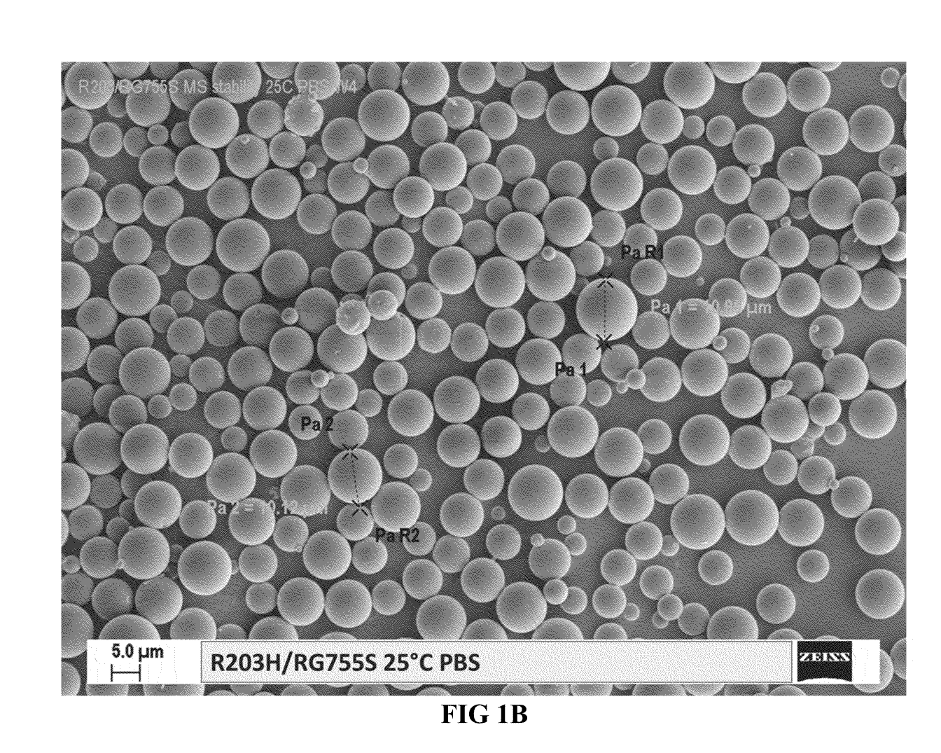

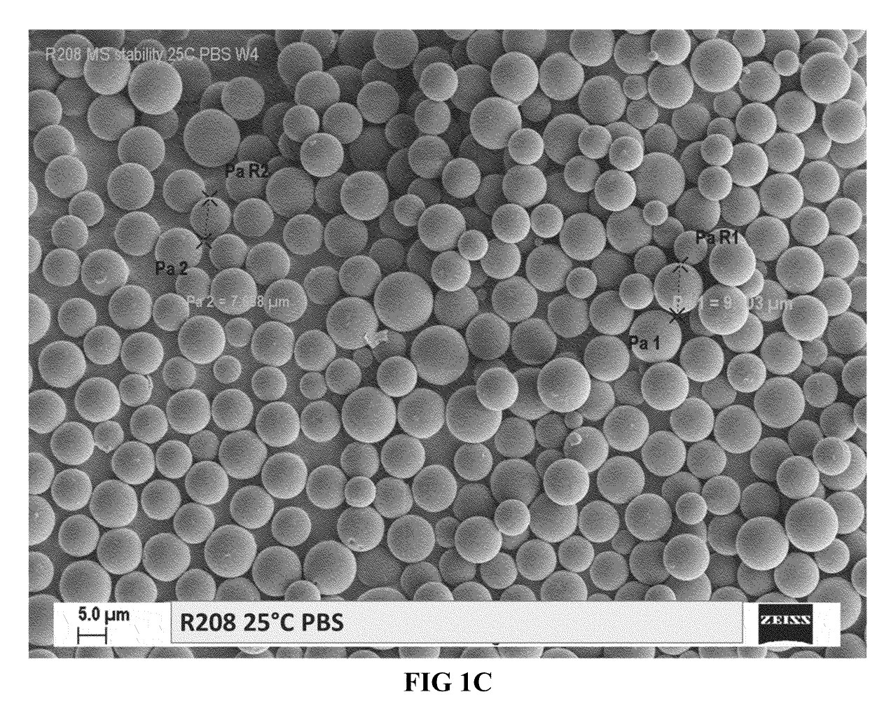

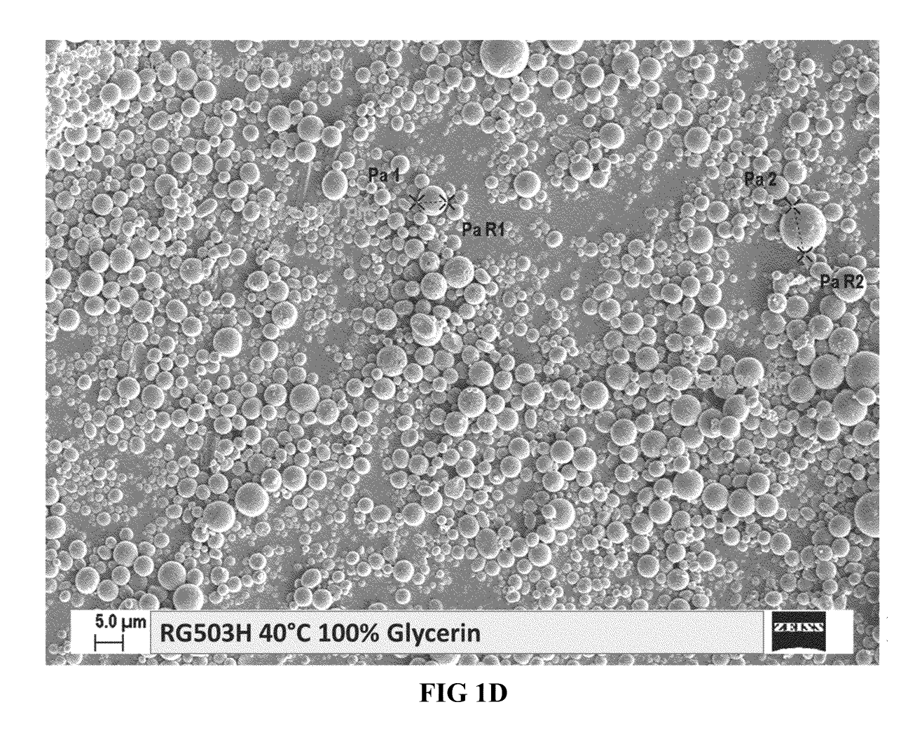

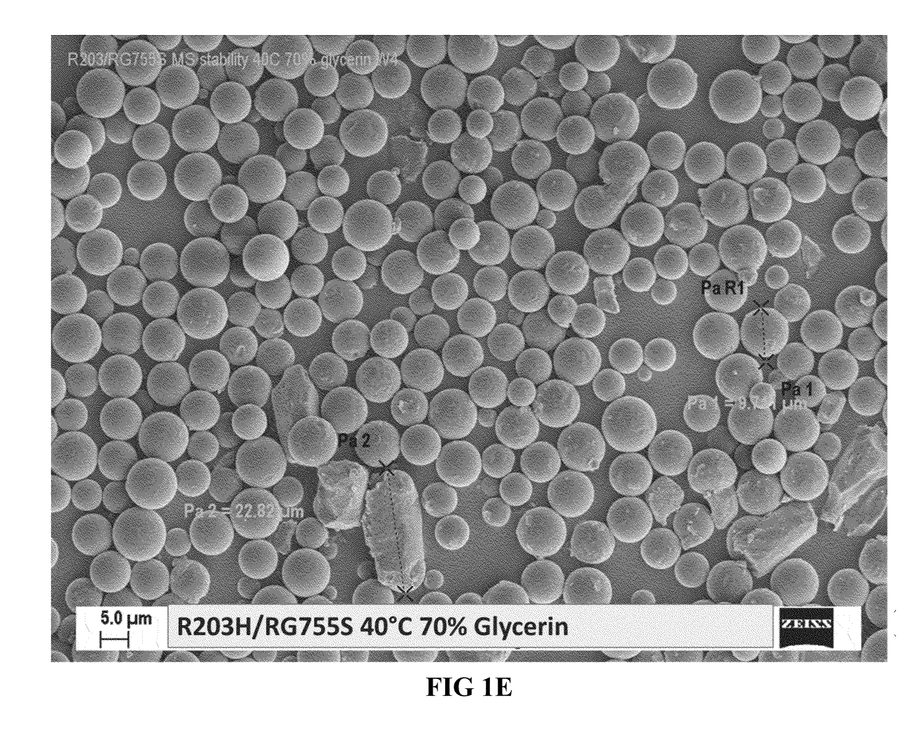

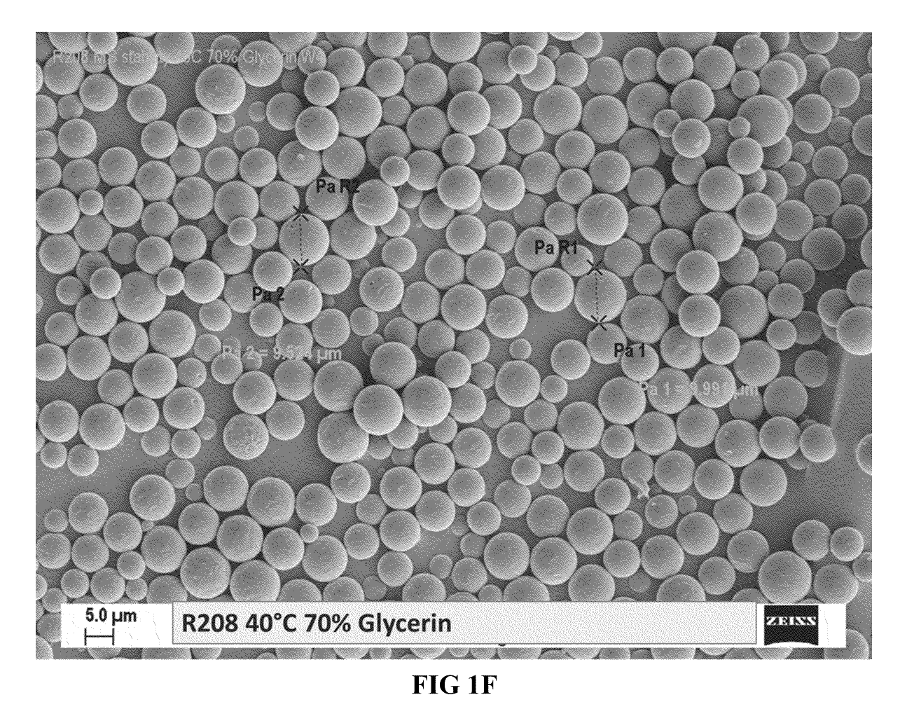

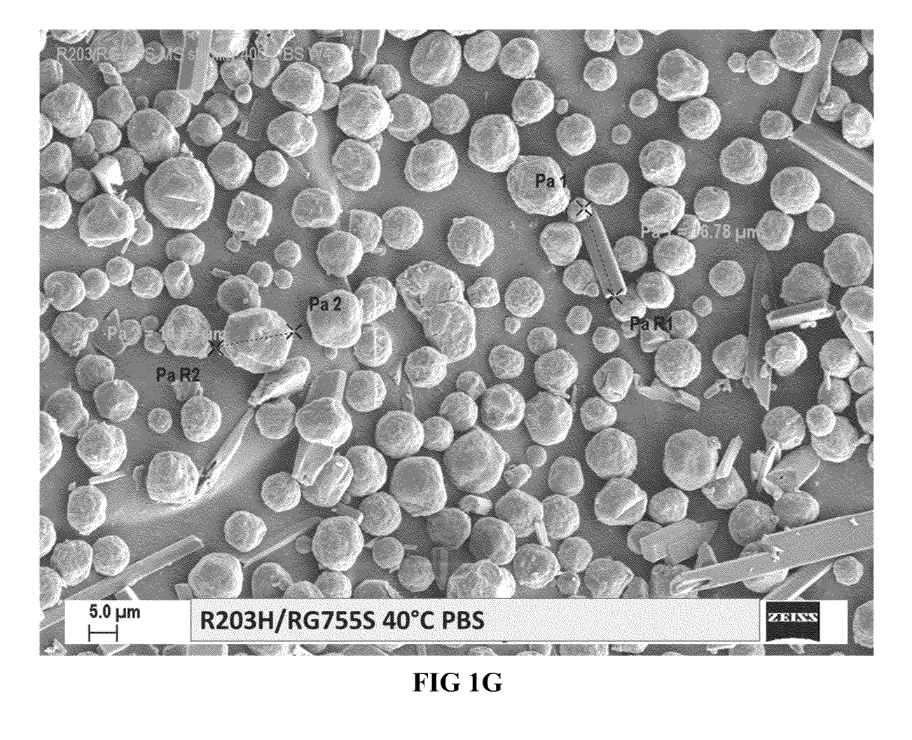

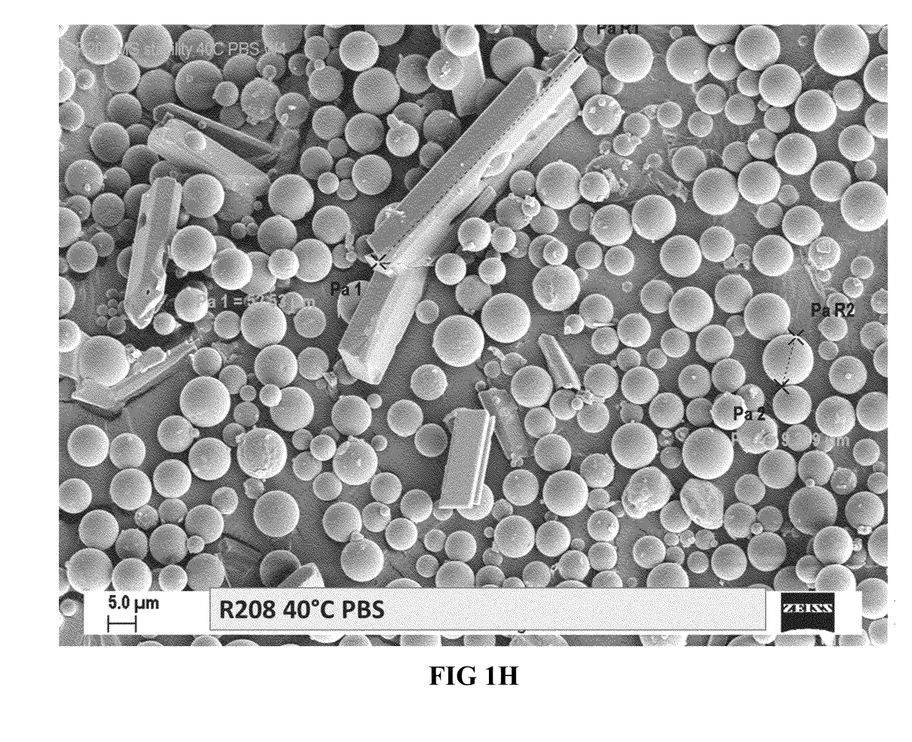

It was found that degradation of PLGA MS when incorporated into a topical aqueous vehicle can be controlled by using the appropriate co-solvent in the topical vehicle base and by storage at appropriate temperature. As shown by FIGS. 1, 2, 3 and 4, the effects of co-solvent (glycerin) in the vehicle and temperature on the stability of incorporated PLGA MS are polymer-dependent.

Thus FIGS. 1A-1H are a collection of eight SEM images for PLGA Tazarotene MS dispersed in phosphate buffered saline (“PBS”) or glycerin vehicles for 1 month.

In FIGS. 1A-1H the magnification was 2000× using a ZEISS EVO® 40 SEM (scanning electron microscope, Carl Zeiss AG, Jena, Germany) set at an acceleration voltage of 5.0 kV. The microspheres (“the particles”) were made using the solvent evaporation/extraction method set forth supra. The microspheres were generally spherical and had an average particle size of about 4 μm to about 10 μm. As shown by FIG. 1 the particles were incubated with different vehicles, such as 70% glycerin or PBS for 1 month, and then vacuum dried, and stored at −20° C. before the SEM photography used to generate FIGS. 1A-1H was carried out. The labels on certain of the particles in the Figures show the type of polymer or polymers used to make the particles, the storage temperature and the vehicle used to store the particles. This experiment determined that the polymer degradation and formation of free drug crystals were highly dependent on the polymer used, vehicles used, and storage temperature used and that the R208 polymer and use of 70% glycerin provided the best results, as well as little particle aggregation.

The labels at the bottom of each of the eight FIG. 1 SEM image shows the specific polymer used to make MS, and the vehicles used for the degradation study. The biodegradable RESOMER® polymers used to make the particles, including RG503H, RG755S, R203H and R208, were obtained from Evonik Industries AG (Germany). The microspheres were prepared by solvent evaporation/extraction method and had an average particle size ranging from 4-10 μm. 5-10 mg of the MS were weighed into a 10 ml glass vial and suspended in vehicles such as phosphate buffer (pH 7.4, made from phosphate buffered saline (“PBS”) from Sigma-Aldrich), 100% glycerin (Spectrum, NF grade), or 70% glycerin solution in water (w/w), as shown in the labels of each SEM image. These glass vials were sealed and incubated in a reciprocal shaking water bath (Precision, Thermo Electron Corporation), which were preset at 25 and 40° C., respectively, and rotated at 45 rpm. After 1 month incubation, the MS were collected via centrifugation, vacuum dried and stored at −20° C. before SEM images were taken.

FIGS. 1A-1H show that RG503H MS degraded in PBS after 1 month incubation at 25° C., while R203H/RG755S combo MS showed degradation only after incubation at 40° C. PBS (see, e.g. FIGS. 1A, 1B, and 1G). Under same conditions, R208 MS degraded the slowest, followed by the R203H/RG755S combo MS, while RG503H MS showed the fastest degradation. Use of 100% or 70% glycerin prevented the degradation of RG503H MS, and R203H/RG755S combo MS, respectively. When MS were incubated in 40° C. PBS buffer, drug crystals were observed for all the three MS, but incorporation of glycerin (70% glycerin) mitigated the formation of drug crystals outside the MS. This was observed more pronouncedly in R208MS, which were incubated at 40° C.

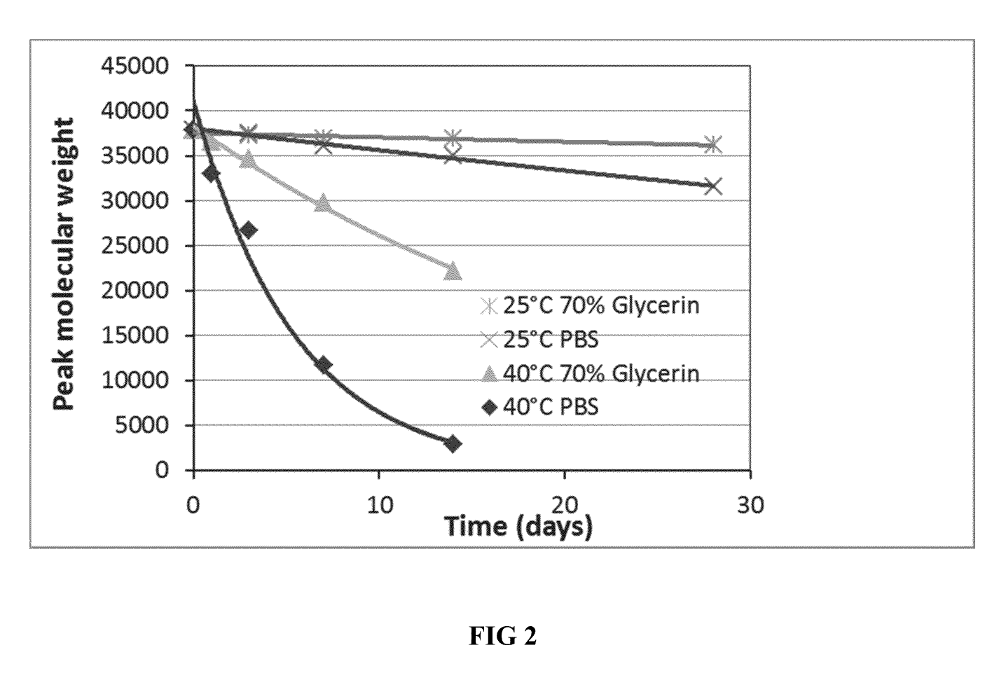

FIG. 2 is a graph showing on the x axis the time in days (up to 28 days), for 2 out of the 3 vehicle conditions since placement of the tazarotene MS identified as “RG503H” in two different vehicles and at two different vehicle concentrations, at two different temperatures, and on the y axis peak molecular weight. The RG503H MS were made using the solvent evaporation/extraction method and comprised 85% polymer and 15% tazarotene), with a mean particle size at 7.7 μm and a drug loading of 15% tazarotene. FIG. 2 shows that the 70% glycerin at 25° C. vehicle showed the least MS degradation, that is the 70% glycerin vehicle reduced the degradation of MS as compared to use of the PBS vehicle.

RG503H Resomer biodegradable polymer was provided by Evonik Industries AG (Germany). MS were prepared by solvent evaporation/extraction method at Evonik with a mean particle size at 7.7 μm and a drug loading of 15% tazarotene. 5-10 mg MS were weighed into a 10 ml glass vial and suspended in vehicles such as phosphate buffer (pH 7.4, made from the phosphate buffered saline from Sigma-Aldrich), or 70% glycerin solution (NF grade glycerin purchased from Spectrum) in water (w/w). These glass vials were sealed and incubated in a reciprocal shaking water bath (Precision, Thermo Electron Corporation), which were preset at 25 and 40° C., respectively, and rotated at 45 rpm. After 4 weeks incubation, MS were collected via centrifugation, vacuum dried and stored at −20° C. before use. MS were dissolved in tetrahydrofuran (THF), sonicated and filtered before loaded into HPLC vials for GPC analysis. GPC was performed with Waters 2690 Separation Module equipped with a Waters 2414 Refractive Index detector. Polystyrene standards were used to calculate the peak molecular weight by the Waters Empower system.

RG503H MS was tested for 28 days with two different vehicles (70% glycerin and PBS). RG503 MS degraded fast, and a clear trend has been observed within one month. FIG. 2 shows that use of 70% glycerin instead of PBS, and use of low temperature 25° C. instead of 40° C. can effectively prevent the degradation of MS.

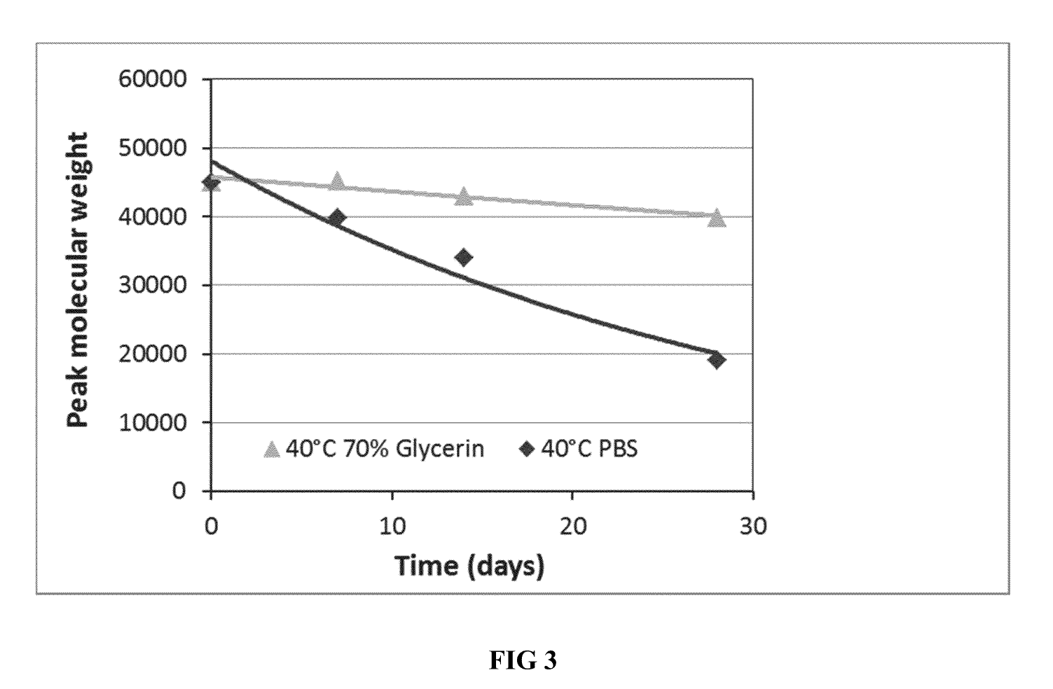

FIG. 3 is a graph showing on the x axis the time in days (up to 28 days) since placement of the tazarotene MS mixture identified as “R203H/RG755S” in two different vehicles and at different temperatures, and on the y axis peak molecular weight. The MS were made using the solvent evaporation/extraction method and their composition was 85% polymer and 15% tazarotene), with a mean particle size of about 5.6 μm and a drug loading of 14.7% tazarotene. FIG. 3 shows that the 70% glycerin vehicle at 40° C. had the least MS degradation, and by comparing with R503H MS, R203H/RG755S MS degraded slower in the same vehicle.

A similar process for RG503H MS was used to make R203H/RG755S MS. R203H and RG755S MS were provided by Evonik Industries AG (Germany). MS were prepared by solvent evaporation/extraction method at Evonik with a mean particle size at 5.6 μm and a drug loading of 14.7% tazarotene. 5-10 mg MS were weighed into a 10 ml glass vial and suspended in vehicles such as phosphate buffer (pH 7.4, made from the phosphate buffered saline from Sigma-Aldrich), or 70% glycerin solution (NF grade glycerin purchased from Spectrum) in water (w/w). These glass vials were sealed and incubated in a reciprocal shaking water bath (Precision, Thermo Electron Corporation), which were preset at 40° C., respectively, and rotated at 45 rpm. After 4 weeks incubation, MS were collected via centrifugation, vacuum dried and stored at −20° C. before use. MS were dissolved in tetrahydrofuran (THF), sonicated and filtered before loaded into HPLC vials for GPC analysis. GPC was performed with Waters 2690 Separation Module equipped with a Waters 2414 Refractive Index detector. Polystyrene standards were used to calculate the peak molecular weight by the Waters Empower system.

R203H/RG755S MS were tested for 28 days with two different vehicles (70% glycerin and PBS). R203H/RG755S MS degraded faster in PBS compared to in 70% glycerin. A comparison of FIG. 3 and FIG. 2 showed that R203H/RG755S MS degraded slower that RG503H MS. This result showed that 70% glycerin is still a better vehicle than PBS in preventing the degradation of R203H/RG755S polymer.

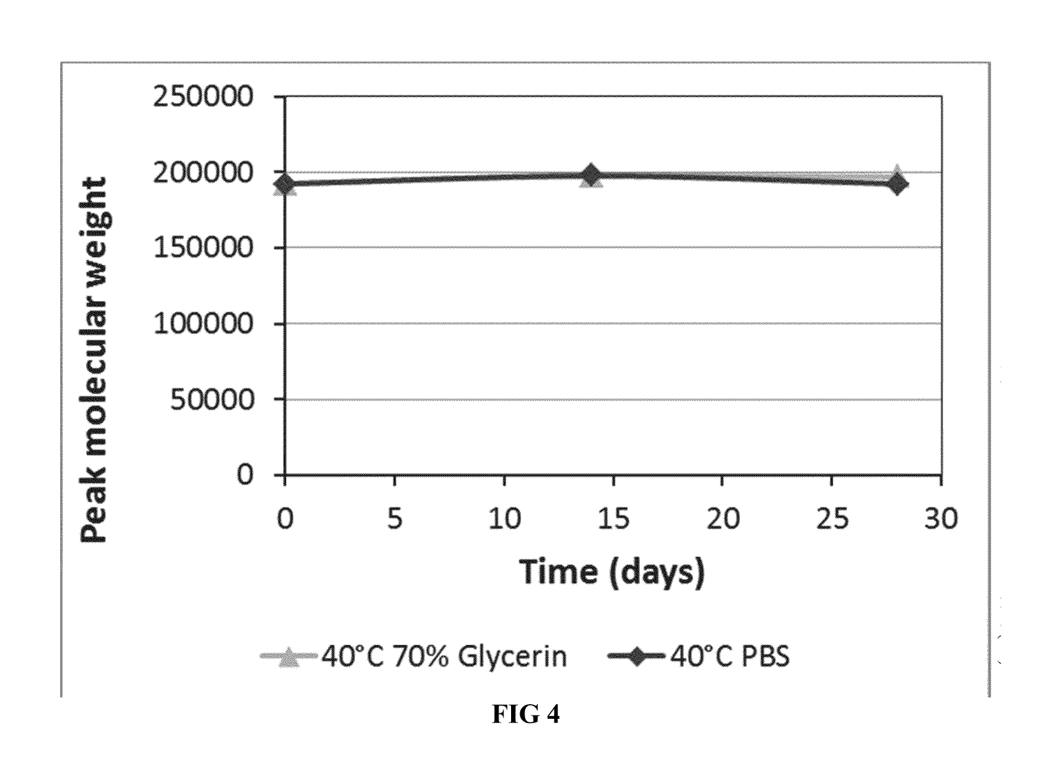

FIG. 4 is a graph showing on the x axis the time in days (up to 28 days) since placement of the tazarotene MS identified as “R208” in two different vehicles and at different temperatures, and on the y axis peak molecular weight. The R208 MS are were made using the solvent evaporation/extraction method, their composition was 85% polymer, and 15% tazarotene), mean particle size was about 6.6 μm and with a drug loading of 14.5% tazarotene). FIG. 4 shows that the vehicles are equivalent at 40° C. because each had the same MS degradation rate. Upon comparing FIGS. 2, 3 and 4, it was discovered that the R208 polymer has the slowest degradation rate, and that use of 70% glycerin significantly slowed down the polymer degradation.

Similar process for RG503H MS was used to make R208 MS. R208 MS were provided by Evonik Industries AG (Germany). MS were prepared by solvent evaporation/extraction method at Evonik with a mean particle size at 6.6 μm and a drug loading of 14.5% tazarotene. 5-10 mg MS were weighed into a 10 ml glass vial and suspended in vehicles such as phosphate buffer (pH 7.4, made from the phosphate buffered saline from Sigma-Aldrich), or 70% glycerin solution (NF grade glycerin purchased from Spectrum) in water (w/w). These glass vials were sealed and incubated in a reciprocal shaking water bath (Precision, Thermo Electron Corporation), which were preset at 40° C., respectively, and rotated at 45 rpm. After 4 weeks incubation, MS were collected via centrifugation, vacuum dried and stored at −20° C. before use. MS were dissolved in tetrahydrofuran (THF), sonicated and filtered before loaded into HPLC vials for GPC analysis. GPC was performed with Waters 2690 Separation Module equipped with a Waters 2414 Refractive Index detector. Polystyrene standards were used to calculate the peak molecular weight by the Waters Empower system.

R208 MS were tested for 28 days at two different vehicles (70% glycerin and PBS). Nearly no degradation was observed in both PBS and 70% glycerin. A comparison of FIGS. 1,2 and 3 showed that R208MS seemed to be the most stable compared to R203H/RG755S MS and RG503H MS.

All of FIGS. 1-4 show that 70% glycerin can prevent the degradation of the PLGA polymers and can be used as the carrier to achieve a long shelf life of the MS. Among the selected the polymers, R208 was the most stable.

In general, it was determined that the non-aqueous solvents reduce water activity, therefore inhibiting the hydrolysis and surface erosion of the PLGA microspheres. However, these solvents can penetrate into the MS matrix, replacing water for solvation. The interaction between co-solvents and polymer is therefore determined on a case-by-case basis. The Applicant has found that this type of interaction is unique and the application for PLGA formulation development is novel. In this Example 1 it was found that glycerin serves a very suitable vehicle co-solvent for PLGA microspheres loaded with tazarotene. In a short list of the co-solvents accepted for dermal products, glycerin significantly reduces the hydrolysis of PLGA but does not increase free drug concentration due to the low solubility of tazarotene in glycerin. Other water miscible co-solvents, such as propylene glycol, PEG 400, ethanol and transcutol, also reduce water activity and PLGA degradation.

### Control of Tazarotene Crystal Formation in Vehicle

It was determined that the formation of tazarotene crystals in a TazMS formulation vehicle can be inhibited by using appropriate co-solvents in the vehicle. Tazarotene loaded into MS is likely at high energy state (possible amorphous state). Thus when water penetrates into the MS and hydrates the MS this then decreases the tazarotene solubility in the PLGA matrix and increases the mobility of tazarotene molecules. When the tazarotene loaded into the MS is dissolved and diffuses out of the MS, the tazarotene will form crystals in the aqueous phase. Hence to prevent tazarotene crystal formation the Applicant has found that a co-solvent can be added to the vehicle to change the solubility of the tazarotene both in the MS matrix and in aqueous phase of the vehicle. Specifically it was found that glycerin and PEG 400 inhibit the crystal formation while transcutol accelerates the crystal formation. In addition, the crystal nucleation and growth are a function of viscosity and surface activity on the solid-liquid interface, the excipients that increase viscosity and reduce surface tension also reduce the formation of drug crystals. It was found that the crystal formation is much slower in a viscous gel containing surfactant than that in an aqueous suspension.

Table 1 shows the solubility of tazarotene in different solvents at different temperatures.

TABLE 1
Tazarotene solubility in water, glycerin, and 70%
glycerin solution

| | Solubility (μg/mL) | | |
|---|---|---|---|
| T ° C. | Water | 70% Glycerin | Glycerin |
| 5 | 0.07 | 0.27 | 72.6 |
| 25 | 0.04 | 0.68 | 34.7 |
| 40 | 1.70 | 4.43 | 526.8 |

The co-solvent effect on tazarotene crystal formation was studied and it was determined that the formation of tazarotene crystal is much faster in the presence of transcutol but not when the transcutol is combined with other non-aqueous co-solvent.

Control of Tazarotene Release from TazMS

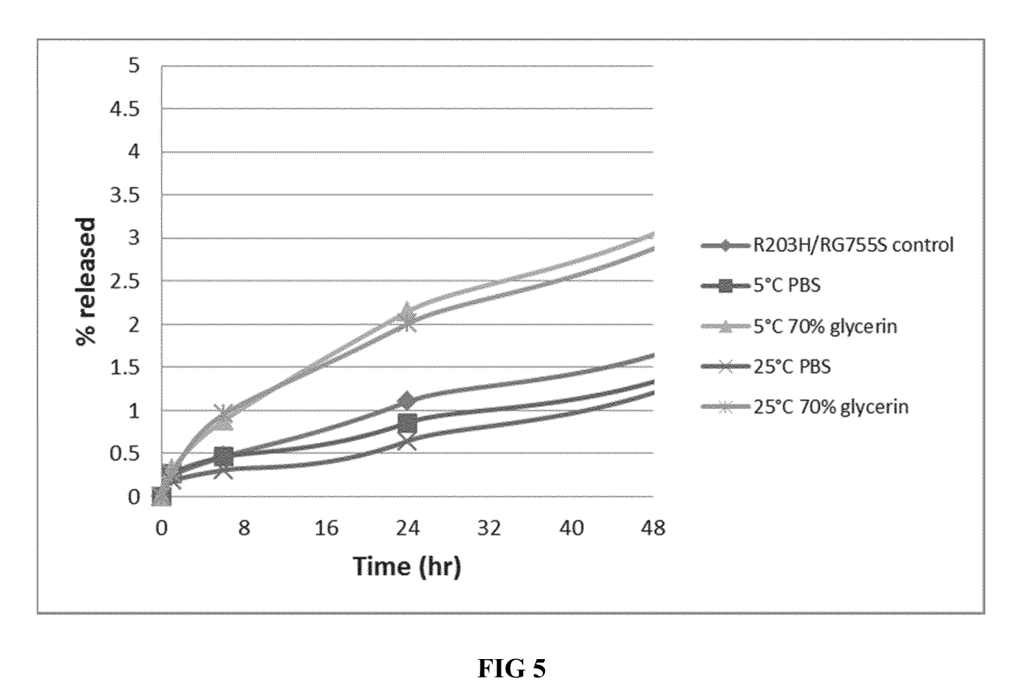

Finally in this Example 1 it was found that release of tazarotene from the MS into isopropyl myristate (IPM) is associated with the co-solvent used in the vehicle. Co-solvents can penetrate into MS matrix, replace water for solvation, and then change drug solubility, solid form and drug distribution in the MS, resulting changes of drug release profile. In a specific example shown in FIG. 5, the tazarotene releases from the MS stored in 70% glycerin is much faster than that from the MS stored in PBS at 5° C. and 25° C. for 1 month.

FIG. 5 is a graph showing on the x axis the time in days (up to 48 hours) since placement of the tazarotene MS mixture identified as “R203H/RG755S” The MS comprised 85% polymer and 15% tazarotene), with a mean particle size at 5.6 μm and a drug loading of 14.7% tazarotene). FIG. 5 shows that the vehicles are roughly equivalent at either temperature (because each had about the same MS degradation). The 25° C. temperature was maintained by incubation in a reciprocal shaking water bath (Precision, Thermo Electron Corporation), which were preset at 25° C.

As was previously done, the solvent/evaporation process was used to make the RG503H, R203H/RG755S MS. R203H and RG755S MS. The MS had a mean particle size at 5.6 μm and a drug loading of 14.7% tazarotene. 5-10 mg MS were weighed into a 10 ml glass vial and suspended in vehicles such as phosphate buffer (pH 7.4, made from the phosphate buffered saline from Sigma-Aldrich), or 70% glycerin solution (NF grade glycerin purchased from Spectrum) in water (w/w). These glass vials were sealed and incubated in a reciprocal shaking water bath (Precision, Thermo Electron Corporation), which were preset at 25° C., and rotated at 45 rpm. Some vials were incubated in a cold room set at 5° C., and were shaken at 45 rpm. After 4 weeks incubation, MS were collected via centrifugation, vacuum dried and stored at −20° C. before use.

2-5 mg vacuum dried MS were weighed into a 1.5 ml centrifuge tube and suspended in 1.0 ml pre-warmed 37° C. isopropyl myristate (IPM, Sigma-Aldrich). These centrifuge tubes were incubated in a reciprocal shaking water bath (Precision, Thermo Electron Corporation), which were preset at 37° C. and rotated at 45 rpm. After different time points, tubes were centrifuged (5000 rpm*3minutes), and supernatants were collected. Fresh IPM of same volume was added, and tubes were resumed to incubation. IPM supernatant was diluted 5 folds by using acetonitrile/isopropyl alcohol (2:1 v/v) and then measured by using HPLC (2690 Separation module, Waters). The mobile phase consisted of 81% acetonitrile and 19% phosphate buffered saline (pH3, 15 mM). Cumulative drug release rate was calculated based on the percentage of drug released compared to the total drug encapsulated within the microspheres.

FIG. 5 shows that the incubation of R203H/RG755S MS with 70% glycerin slightly increased the drug release rate at the initial period, while the use of PBS did not affect much on the drug release rate. It is noted that at 5 and 25° C., not much free drug crystals was formed after incubation of MS with the vehicles. However, at higher temperature or use of RG503H MS may facilitate the formation of free drug crystals in the exterior of the MS, drug release rate profile may behave differently.

### Example 2

### Determination of Polymeric Tazarotene Delivery Systems for Targeting Pilosebaceous Units

In this Example 2 polymeric particulate systems for follicular delivery of tazarotene were studied including the study of particles with the average particulate size of about 0.1 microns to about 10 microns, with a tazarotene loading in the microspheres of from about 1% by weight to about 90% by weight, and with from about 10% to about 80% of the tazarotene released from the microparticles within about 24 hours drug release rate into a sebum-like vehicle. The polymer comprising the polymeric particulate system can include biodegradable and non-biodegradable polymers, such as PLGA, polystyrene, polymethylacrylates, and ethyl cellulose.

A novel follicular delivery system using tazarotene as the active pharmaceutical ingredient (“API”) for the treatment of various dermatological conditions, including acne has been developed. An improved efficacy and reduced skin irritation, in hamster models, of the tazarotene loaded PLGA microspheres (MS) has been demonstrated.

### Polymer and MS Structure Selection

The Applicant has found that tazarotene is compatible with various polymers used to make MSs but that the morphologic property and the performance of these MS are substantially different, depending on the polymer type and manufacturing process used. The investigation of the polymer selection demonstrated that tazarotene can be encapsulated in various polymers, biodegradable (PLGA) and non-biodegradable (ethyl cellulose and polystyrene) polymers, and with various morphologic structures (matrix and porous).

The rational for polymer selection is set forth in Table 2. These polymers include (1) biodegradable PLGA polymers with different degradation and surface erosion properties in water, as well as lipophilicity, and composition, (2) polystyrene, non-biodegradable polymer, (3) ethyl cellulose, non-biodegradable but more soluble in non-aqueous solvents. The particles size, drug release, stability, and in vivo performance of these MS are summarized in Table 3.

TABLE 2
Polymers Studied

| Polymers | Rational for use to make Tazarotene Microspheres |
|---|---|
| RG 503H | Fast biodegradable |
| RG 755S | Biodegradable |
| | Relatively lipophilic polymer (ester end) |
| R208 | Slow biodegradable polymer, (PLA) |
| | Long shelf-life in aqueous vehicles |
| R203H/RG755S 1:1 | Biodegradable |
| | Combination of relatively lipophilic and |
| | hydrophilic polymers |
| | Burst effect |
| R203H/RG502H 1:3 | Biodegradable |
| | Combination of relatively lipophilic and |
| | hydrophilic polymers |
| | Burst effect |
| Ethyl cellulose | Non-biodegradable |
| | Soluble in most organic solvents |
| | Stable in aqueous vehicles |
| Polystyrene | Non-biodegradable |
| | Not soluble in most organic solvents |
| Polystyrene, | Non-biodegradable |
| macroporous MS | Not soluble in most organic solvents |
| | Relatively fast drug release from MS |

TABLE 3
Summary of Tested TazMS

| | | | | | | | | 6-w MS | | |
|---|---|---|---|---|---|---|---|---|---|---|
| | | | % PS | | | | | stability | | |
| | | Mean | Fraction | | | | Polymer | (potency | | |
| | | PS, | in 2-7 | % Release | | | stability | release, | | |
| Lot | Polymer | μm | μm | T = 0 h | T = 1 h | T = 24 h | in water* | PS) | Efficacy | Irritation |
| 737-33 | RG 503H | 7.7 | 42 | 6.4 | 6.7 | 7.2 | Poor | OK | ++ | ++ |
| 737-61 | RG 755S | 6.3 | 29 | 0.6 | 0.4 | 1.5 | Fair | OK | | |
| 737-40 | R208 | 6.6 | 60 | 1.0 | 1.3 | 2.7 | Good | OK | | |
| 737-43 | R203H/ | 5.6 | 43 | 0.9 | 0.7 | 1.8 | Fair | OK | 0 | + |
| | RG755S 1:1 | | | | | | | | | |
| 737-71 | R203H/ | 4.3 | 55 | 4.5 | 2.8 | 3.0 | Fair | OK | | |
| | RG502H 1:3 | | | | | | | | | |
| 737-50 | Ethyl | 10.1 | 22 | 65.4 | 95.9 | 95.3 | Excellent | OK | | |
| | cellulose | | | | | | | | | |
| 737-53 | Polystyrene | 9.1 | 29 | 60.6 | 93.3 | 97.5 | Excellent | OK | | |
| 737-78 | Polystyrene, | 15.6 | 24 | 46.4 | 76.5 | 102.7 | Excellent | Low | ++++ | ++++ |
| | macroporous | | | | | | | potency | | |
| | MS | | | | | | | | | |

### Drug Release Profile

It was determined that the tazarotene release profile from tazarotene containing microspheres into isopropyl myristate (IPM) (an artificial sebum) is an indication of tazarotene distribution in the matrix of and on the surface of the MS. Thus it provides a predictive parameter for the in vivo performance of the MS in a hamster flank model.

The efficacy and irritation results shown in Table 4 show that the tazarotene release profiles from TazMS is an important factor in the design of the delivery system and control of TazMS's performance in vivo. Within the desirable range of particle size, the tazarotene release in IPM (an artificial sebum) is preferably greater than about 15% of the tazarotene loaded in the MS (0.15 mg/mL of final formulation) at 24 hr to achieve significant efficacy, and less than about 50% of the tazarotene loaded in the MS at 0 to plus one hour in order to avoid significant irritation. Therefore, a release profile of about 15% to about 80% of the tazarotene released by 24 hours and with a sustained release rate is desirable to improve efficacy and reduce skin irritation. It was further determined that the type of polymer and the structure of the microspheres does not have a critical role in the in vivo performance of the MS.

TABLE 4
Taz release from MS vs. in vivo performance

| | | % Release in IPM | | | |
|---|---|---|---|---|---|
| TazMS Lot | Polymer | 0 hr | 24 hr | Efficacy | Irritation |
| 737-33 | RG 503H | 6.4 | 7.2 | ++ | + |
| 737-40 | R208 | 1.0 | 2.7 | | |
| 737-43 | R203H/RG755S | 0.9 | 1.8 | 0 | 0 |
| | 1:1 | | | | |
| 737-50 | Ethyl cellulose | 65 | 95.3 | | |
| 737-53 | Polystyrene | 61 | 97.5 | | |
| 737-78 | Polystyrene, | 46 | 102.7 | ++++ | ++++ |
| | macroporous | | | | |
| | MS | | | | |
| AGN-2, 7.3 μm | RG 503H | | 37 (IPM), 33 & 44 | ++++ | ++ |
| | | | (sebum) | | |
| AGN-1, 4.2 μm | RG 503H | | 25 & 58 (sebum) | ++++ | +++ |
| Nanomi-1, | RG 503H | 60 | 97 | ++++ | ++++ |
| 0.57 μm | | | | | |
| Nanomi-1, 2.1 μm | RG 503H | 37 | 45 | NA | NA |
| Nanomi-1, 6.5 μm | RG 503H | 23 | 24 | ++++ | ++ |
| Nanomi-1, 10 μm | RG 503H | 5 | 8 | NA | NA |

This optimal release profile for tazarotene applies to retinoids in general and was an unexpected and unpredicted discovery.

Additionally, defining drug release profile for the quality control of MS and DP (drug product), and use to predict performance in vivo (i.e. humans) is a novel method.

### Drug Loading in MS

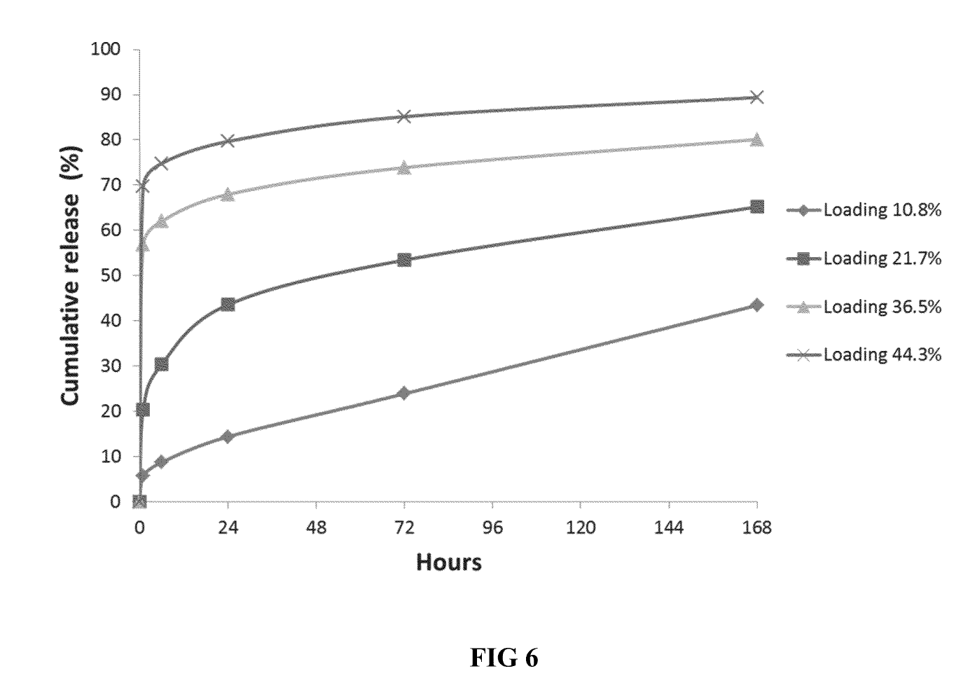

It was found that the drug release profile from TazMS can be controlled by drug-loading within a specific range for a specific polymer. Drug release from MS is associated with multiply factors. The physicochemical properties of APIs and polymers, the physical and chemical interaction between an API and a polymer, the morphologic structure of MS, the distribution and solid state of APIs in MS, the composition and preparation process of MS, the residual solvent, the additives, and the vehicles. Drug-loading in MS is also one of the critical factors to impact the drug release profile. The effect of tazarotene loading in R208 MS were investigated and the results are shown in FIG. 6.

Additionally, as shown by Table 5 the higher drug loading in the tested PLGA TazMS increased the drug release rate. Although SEM images showed that a small amount of drug crystals in the MS samples, the fraction of the crystals will not significantly affect the in vitro and in vivo performance as demonstrated by the slow release rate; less than 3% of the tazarotene released from these particular microspheres in the first hour after incubation with isopropyl myristate at 37° C. The R208 TazMS at 28.9% drug loading showed a preferred drug release profile from microspheres with a preferred particle size distribution. With good biocompatibility with vehicles and the noted desirable drug release profile, R208 TazMS with about 30% drug loading in the microspheres is a desirable embodiment of the sustained release tazarotene microparticles formulation for the treatment of a dermatological condition. The formulation of this desirable embodiment is R208 (PLA) microspheres with about 30% tazarotene loading and with a 6.6 μm microparticles size distribution, in isopropyl myristate vehicle.

The drug-loading study conducted is shown in Table 5.

TABLE 5
Summary of drug-loading study

| | | Particle size | | | | | | | | | |
|---|---|---|---|---|---|---|---|---|---|---|---|
| | | | | | % | | | | | | |
| | | | | | in | | | | | | |
| | % | Mean | D90 | D10 | 2-7 | % Drug release | | | | | |
| Polymer | API | (μm) | (μm) | (μm) | μm | 0 hr | 1 hr | 2 hr | 4 hr | 6 hr | 24 hr |
| R208 | 28.9 | 6.4 | 8.6 | 4.5 | 66 | 1.1 | 2.8 | 3.3 | 4.8 | 5.9 | 23.6 |
| R208 | 19.4 | 6.4 | 8.4 | 4.8 | 69 | 0.7 | 1.1 | 1.2 | 1.7 | 2.0 | 5.7 |
| R208 | 14.5 | 6.6 | 8.6 | 4.9 | 60 | 1.0 | 1.3 | 1.2 | 1.3 | 1.5 | 2.7 |
| R203H/RG755S | 47.3 | 8.2 | 10.8 | 5.7 | 30 | 2.7 | 5.6 | 5.7 | 6.3 | 6.5 | 8.2 |
| R203H/RG755S | 28.3 | 6.9 | 9.0 | 0.5 | 49 | 1.0 | 1.7 | 1.8 | 2.1 | 2.3 | 5.5 |
| R203H/RG755S | 19.1 | 6.3 | 8.2 | 4.6 | 70 | 1.0 | 1.3 | 1.5 | 1.9 | 2.2 | 4.8 |
| R203H/RG755S | 14.7 | 5.6 | 8.9 | 0.5 | 43 | 0.9 | 0.7 | 0.9 | 0.6 | 0.8 | 1.8 |

Surprisingly, the drug-loading effect on the release for TazMS is significantly dependent upon the polymer used to make the microspheres and the level of tazarotene loading in the microspheres. The drug release from R203H/RG755S MS increases from 1.8% to 8.2% within 24 hrs when the drug-loading is up from 15% to about 50%, while the release from R208 MS increases from 2.7% to 24% within 24 hrs when drug-loading is doubled from 15% to about 30%. This is an unexpected and unpredicted discovery because it was expected that increasing drug loading will increase the drug release rate due to the increased drug gradient. Furthermore, increasing drug loading will increase the concentration of the drug outside the microspheres, leading to increased initial burst effect release of the tazarotene from the MS.

It was determined that a dramatic increase of the amount of tazarotene released at high amount of tazarotene loading in the microspheres is presumably associated with the solid state change from the amorphous to the crystal form of the drug) This conclusion was based upon four experiments (A-D) summarized below which examined the DSC (differential scanning calorimetry) for solid state tazarotene in the R208 MS at either a low and high drug-loading in the microspheres.

A. With pure tazarotene crystal the melting point was 104° C., the heat of fusion was 89.64 J/g and amorphous tazarotene formed upon cooling from its melted state.

B. With a mixture of Taz crystal and RG755 the mixture had an 18% w/w of the tazarotene in the mixture and based on peak area, the calculated weight percentage of the tazarotene I the mixture was 15.80(J/g)/89.64(J/g)=17.6%.

C. With R208 MS with 15% drug loading no melting peak was observed and the form of the tazarotene was amorphous.

D. Finally, R208 MS with 30% drug loading: if all the drug is crystalline, peak area should be 89.64(J/g)*30%=26.9 J/g. Hence the percentage of drug that is crystal in MS is 2.16/26.9=8%.

### Example 3

### Tazarotene Microspheres for Follicular Drug Delivery

In this Example 3 it was determined that polymeric particulate systems for follicular delivery of tazarotene can have an average particulate diameter of from about 0.1 microns to about 10 microns, with the tazarotene loaded therein at from about 1% to about-90%. The 1% to 90% is the percentage of the drug encapsulated in MS, that is drug weight/total weight of MS×100%, determined by weighing the MS, then dissolving the MS in an organic solvent, and determining the drug amount using HPLC analysis. The MS released from about 10% to about 80% of the tazarotene (as percentage of the drug compared to the total amount of drug encapsulated into MS) within 24 hours after being suspended in isopropyl myristate (i.e. in a sebum-like vehicle).

Transdermal retinoid delivery through follicular pathway has important advantages. For example not only does it improve drug bioavailability through the targeted delivery, it can also eliminate much of the side effects, such as irritation, that may be caused by skin penetration.

It was determined that tazarotene loaded microsphere useful for follicular penetration desirably have microsphere diameters between about 2 microns to about 7 microns. Additionally, it was determined that tazarotene microsphere with diameters less than about 2 microns drug release the tazarotene very quickly onto the surface of the skin to which the MS formulation is applied, and that skin absorption thereafter of the quickly released tazarotene can cause considerable skin irritation. It was also determined that microspheres with submicron diameters tend to aggregate, hence this Example 3 studied determined that the desirable average MS diameters for effective sustained follicular delivery of therapeutically effect amounts of the tazarotene loaded in the MS, with little or no skin irritation resulting, are from about 2 microns to about 7 microns (with the upper D90 being less than about 10 microns, and the lower D10 being greater than about 0.5 microns), with the percent of a population the tazarotene loaded microsphere with diameters between about 2 microns to about 7 microns is at least about 30%. It was determined that microparticle diameters greater than about 7 microns are too large for effective follicle penetration by the MS, that is with diameters greater than about 7 microns fewer particle penetrate into follicles and the follicle penetration depth tends to be shallow.

Thus, it was determined that desirable polymeric particulate systems for follicular delivery of tazarotene can have an average particulate diameter of from about 0.1 microns to about 10 micron, rug loading of from about 1% to about 90%, and a drug release rate of from about 10% to about 80% drug release within about 24 hours in sebum-like vehicles.

### Experimental

Monodispersed microspheres with different particle sizes were made by the Nanomi microsieve emulsion process. Thus drug and polymer were dissolved into the organic solvent ethyl acetate. Monodispersed droplets were generated by dispersing this solution into aqueous phase through precise microsieves. Microspheres with very narrow particle size distribution were produced after these droplets solidified.

Tazarotene loaded microspheres at particle sizes of 0.5, 2, and 10 micron were prepared. Drug loading was about 13%.

### Ex Vivo Skin Penetration Experiments

Pig ears were purchased from Sierra Medical Company, Whittier, Calif. On the day of receipt, the pig ears were cleaned by washing in cold tap water, and dried with paper towels. The hair on the dorsal side of the ear was shaved with a clipper. The shaved pig ear skin was cut into approximately 2-inch squares. The shaved areas were cleaned with water followed by 70% ethanol and dried with paper towel. Areas for treatments were marked with circles by using an 8 mm biopsy punch (Miltex REF 33-37). TazMS were suspended in phosphate buffer saline (PBS) to a final Taz concentration of 0.1%. 20 μL was added to each circle and rubbed into the skin for 2 min with a glass rod at room temperature. The skin was then cleaned with water-wet cotton applicators and dried with Kimwipes. The direction of hair was marked and 8 mm punches were made with a biopsy punch. The cartilage from each skin punch was removed by cutting with a scalpel and discarded. To provide flatness and support for cryosection embedding, the skin punches were placed on 8-mm circles made from index cards and kept in −80° C. freezer. To embed for vertical sectioning of HF, the cut punch on index card was placed with the cut edge toward the bottom of a Cryomold (Tissue-Tek #4566) which was filled with O.C.T (Tissue-Tek #4583) and sitting on a metal plate (Biocision Cool Sink 48) which in turn was placed on a dry ice/ethanol bath. Cryosectioning was done on a Leica CM3050S cryostat. Nine 100 μm sections were placed on a Superfrost Plus Micro Slide (VWR #48311-703). The slides were rinsed once in water, coverslipped and examined under the microscope or stored in −20° C. before examination. Microslides of cyrosections of pig ear skin were examined for fluorescence (340 nm excitation) and brightfield (BF) on an Olympus IX71 microscope, using a 10× objective. Images were recorded using the digital camera system and the SlideBook 5.0 software. The fluorescence and bright field images of full-length HF were superimposed using Adobe Photoshop CS5. A stage micrometer (Microscope Depot S-14710) was also photographed in BF and was used to measure the distance of penetration of TazMS.

This Example 3 study showed that as microsphere diameters increased from 2.1 microns to 10 microns skin penetration ability of the microspheres decreased, and that microspheres with diameters of from about 2.1 microns to about 6.5 microns had the best skin penetration. The 0.57 micron diameter TazMS MS formed large sheets that limited skin penetration.

TABLE 6
Summary of hair follicle penetration profiles of
Taz MS of 4 different sizes

| | | | | Avg. |
|---|---|---|---|---|
| | % Positive | | Avg. Distance | Deepest |
| Microsphere | Follicles/Total | | Concentrated | Distance |
| Size | Follicles | Intensity | Area | Penetration |
| 0.57 μm | 7/10 | + | 373 μm | 743 μm |
| 2.1 μm | 12/14 | ++++ | 529 μm | 1508 μm |
| 6.5 μm | 11/15 | +++ | 663 μm | 1291 μm |
| 10 μm | 10/12 | ++ | 410 μm | 1100 μm |

In the Table 6 above microsphere size was determined using a Malvern Sizer 2000 particle size analyzer; “% positive follicles means the percentage of follicles that have particle penetration; and since the tazarotene present can be made fluorescent, the intensity was measured using a fluorescent microscope.

Based on this Example 3 study, a desireable microsphere particle diameter with this composition and under these conditions is from about 2 microns to about 7 microns.

Further studies were carried out to determine biotolerability (i.e. skin irritation) of tazarotene containing microspheres when administered dermally to the skin of pigs and to the (shaved skin) of hamsters.

Patents and publications mentioned in the specification are indicative of the levels of those skilled in the art to which the invention pertains. These patents and publications are incorporated herein by reference to the same extent as if each individual application or publication was specifically and individually incorporated herein by reference.

While the invention has been described in terms of various specific and preferred embodiments, the skilled artisan will appreciate that various modifications, substitutions, omissions, and changes may be made without departing from the spirit thereof. In particular, the indication of a particular embodiment or parameter as being “preferred” should not be construed as indicating that other embodiments and/or parameters described herein are not desirable. Accordingly, it is intended that the scope of the present invention be limited solely by the scope of the following claims, including equivalents thereof.

We claim:

1. A topical dermal composition comprising:
(a) a plurality of biodegradable polymeric microparticles;
(b) a retinoid contained by the microparticles, and;
(c) a vehicle for the microparticles comprising an aqueous solvent and a non-aqueous solvent.

2. The composition of claim 1 wherein the non-aqueous solvent is selected from the group consisting of glycerin, propylene glycol, ethanol and transcutol.

3. The composition of claim 2 wherein the vehicle is about 70% glycerin and about 30% saline.

4. The composition of claim 1 wherein the retinoid is selected from the group consisting of tretinoin, adapalene, and tazarotene, combination, salts and esters thereof.

5. The composition of claim 1 wherein the microparticles have an average diameter of between about 1 micron and about 10 microns.

6. The composition of claim 1 wherein the microparticles have an average diameter of between about 2 microns and about 7 microns.

7. The composition of claim 1 wherein the biodegradable polymer is selected from the group consisting of polymeric lactic acid, polymeric glycolic acid, and polymeric lactic acid glycolic acid (“PLGA”), and combinations thereof.

8. The composition of claim 7 wherein the biodegradable polymer is PLA.

9. The composition of claim wherein the retinoid loading in the microspheres is about 30%.

10. A topical dermal composition comprising:
(a) a plurality of biodegradable PLGA or PLA microparticles having an average diameter of between about 2 microns and about 7 microns;
(b) tazarotene contained by the microparticles at about 30% drug loading; and
(c) a vehicle for the microparticles comprising saline and glycerin.

11. A method for treating a dermatological condition selected from the group consisting of acne vulgaris, seborrhoeic dermatitis, psoriasis, keratosis pilaris, and photoaged skin, the method comprising the stop of administering to the skin of a person with a dermatological condition a topical dermal composition comprising:
(a) a plurality of biodegradable polymeric microparticles;
(b) a retinoid contained by the microparticles, and;
(c) a vehicle for the microparticles comprising an aqueous solvent and a non-aqueous solvent;
thereby treating the dermatological condition.

12. The method of claim 11, wherein the composition provides for an extended release of the retinoid.

13. A method for treating acne, the method comprising the step of administering to the skin of a person with acne a topical dermal composition comprising:
(a) a plurality of biodegradable PLGA microparticles having an average diameter of between about 2 microns and about 7 microns;
(b) tazarotene contained by the microparticles at about 30% drug loading, and;
(c) a vehicle for the microparticles comprising saline and glycerin, thereby treating the acne.

14. A dermal topical composition comprising
(a) a plurality of biodegradable polymeric microparticles;
(b) a vehicle for the microparticles comprising an aqueous solvent and a non-aqueous solvent, and;
(c) a compound of the following formula contained by or encapsulated by the microparticles: 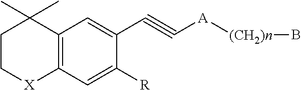
wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4; and
wherein the microparticles have an average diameter between about 0.1 μm and about 10 μm.

15. The composition of claim 14, wherein the microparticles have an average diameter no greater than about 5 μm.

16. The composition of claim 14, wherein the microparticles have an average diameter no greater than about 4 μm.

17. The composition of claim 14, wherein the microparticles have an average diameter no greater than about 1 μm.

18. The composition of claim 14, wherein the biodegradable polymer is selected from the group consisting of poly hydroxyaliphatic carboxylic acids, polyesters, polysaccharides, and combinations thereof.

19. The composition of claim 14, wherein the biodegradable polymer is poly(lactic-co-glycolic acid) (PLGA).

20. The composition of claim 14, wherein the compound is tazarotene.

21. The composition of claim 14, wherein the compound is tazarotenic acid or a pharmaceutically acceptable salt, ester or amide thereof.

22. A method for treating a condition associated with excess sebum production, the method comprising the step of topically applying to the skin of a patient in need of such treatment a dermal composition comprising:
(1) a plurality of biodegradable polymeric micronanoparticles;
(2) encapsulated by or encompassed by the microparticles a compound of the formula: 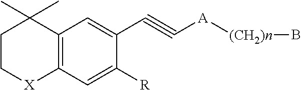
wherein:
X is S, O, or —N(R¹)— where R¹ is hydrogen or lower alkyl;
R is hydrogen or lower alkyl;
A is pyridinyl, thienyl, furyl, pyridazinyl, pyrimidinyl or pyrazinyl;
n is 0-2;
B is selected from the group consisting of: H, —COOH or a pharmaceutically acceptable salt, ester or amide of said —COOH group, —CH₂OH or an ether or ester derivative of said —CH₂OH group, —CHO or an acetal derivative of said —CHO group, and —COR² or a ketal derivative of said —COR² group, wherein R² is —(CH₂)ₘCH₃ wherein m is 0-4;
and;
(3) a vehicle for the microparticles comprising an aqueous solvent and a non-aqueous solvent,
wherein the microparticles have an average diameter between about 0.1 μm and about 10 μm; and wherein the compound penetrates the hair follicle to the depth of the sebaceous gland, and acts directly on the gland to reduce sebum production by the gland.

23. A method for treating a condition associated with excess sebum production, the method comprising the step of topically applying to the skin of a patient in need of such treatment a dermal composition comprising:
(1) plurality of biodegradable, polymeric microparticles
(2) encapsulating by or encompassed by the microparticles a compound of the formula: 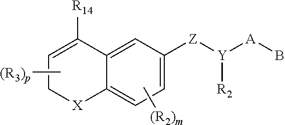
or a pharmaceutically acceptable salt thereof,
wherein:
X is S, O, NR′, where R′ is H or alkyl of 1 to 6 carbons; or X is [C(R₁)₂]ₙ where R₁ is independently H or alkyl of 1 to 6 carbons, and n is an integer between, and including, 0 and 2, and;
R₂ is hydrogen, lower alkyl of 1 to 6 carbons, F, Cl, Br, I, CF₃, fluoro substituted alkyl of 1 to 6 carbons, OH, SH, alkoxy of 1 to 6 carbons, or alkylthio of 1 to 6 carbons, and;
R₃ is hydrogen, lower alkyl of 1 to 6 carbons or F, and;
m is an integer having the value of 0-3, and;
p is an integer having the value of 0-3, and;
Z is —C≡C—, —N═N—, —N═CR₁—, —CR₁═N, —(CR₁═CR₁)_{n′}— where n′ is an integer having the value 0-5, —CO—NR₁—, —CS—NR₁—, —NR₁—CO, —NR₁—CS, —COO—, —OCO—, —CSO—, or —OCS—;
Y is a phenyl or naphthyl group, or heteroaryl selected from a group consisting of pyridyl, thienyl, furyl, pyridazinyl, pyrimidinyl, pyrazinyl, thiazolyl, oxazolyl, imidazolyl and pyrrazolyl, said phenyl and heteroaryl groups being optionally substituted with one or two R₂ groups, or, when Z is —(CR₁═CR₁)_{n′}— and n′ is 3, 4 or 5 then Y represents a direct valence bond between said (CR₂═CR₂)_{n′} group and B;
A is (CH₂)_{q} where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds;
B is hydrogen, COOH or a pharmaceutically acceptable salt thereof, COOR₈, CONR₉R₁₀, —CH₂OH, CH₂OR₁₁, CH₂OCOR₁₁, CHO, CH(OR₁₂)₂, CHOR₁₃O, —COR₇, CR₇(OR₁₂)₂, CR₇OR₁₃O , or tri-lower alkylsilyl, where R₇ is an alkyl, cycloalkyl or alkenyl group containing 1 to 5 carbons, R₈ is an alkyl group of 1 to 10 carbons or trimethylsilylalkyl where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons, or a cycloalkyl group of 5-10 carbons, or phenyl or lower alkylphenyl, R₁₁ is lower alkyl, phenyl or lower alkylphenyl, R₁₂ is lower alkyl, and R₁₃ is divalent alkyl radical of 2-5 carbons, and
R₁₄ is (R₁₅)ᵣ-phenyl, (R₁₅)ᵣ-naphthyl, or (R₁₅)ᵣ-heteroaryl where the heteroaryl group has 1 to 3 heteroatoms selected from the group consisting of O, S and N, r is an integer having the values of 0-5, and
R₁₅ is independently H, F, Cl, Br, I, NO₂, N(R₈)₂, N(R₈)COR₈, NR₈CON(R₈)₂, OH, OCOR₈, OR₈, CN, an alkyl group having 1 to 10 carbons, fluoro substituted alkyl group having 1 to 10 carbons, an alkenyl group having 1 to 10 carbons and 1 to 3 double bonds, alkynyl group having 1 to 10 carbons and 1 to 3 triple bonds, or a trialkylsilyl or trialkylsilyloxy group where the alkyl groups independently have 1 to 6 carbons;
and;
(3) a vehicle for the microparticles comprising an aqueous solvent and a non-aqueous solvent,
wherein the microparticles have an average diameter between about 0.1 μm and about 10 μm; and wherein the compound penetrates the hair follicle to the depth of the sebaceous gland, and acts directly on the gland to reduce sebum production by the gland.
